(19) United States
(12) Patent Application Publication
(10) Pub. No.: US 2006/0134607 A1
(43) Pub. Date: Jun. 22, 2006

(54) Marker gene

(75) Inventors: Masaaki Miyazawa, Osaka (JP); Mario Clerici, Milan (IT)

Correspondence Address: EDWARDS & ANGELL, LLP, P.O. BOX 55874, BOSTON, MA, 02205, US

(73) Assignee: ImmunoClin Limited Osaka Industrial Promotion Organization

(21) Appl. No.: 10/531,193

(22) PCT Filed: Oct. 16, 2003
(86) PCT No.: PCT/GB03/04493

§ 371 Date: Jan. 3, 2006

(30) Foreign Application Priority Data
Oct. 16, 2002 (GB) 0223982.0

(51) Int. Cl. C12Q 1/70; C12Q 1/68

(52) U.S. Cl. 435/5; 435/6

(57) ABSTRACT

A method of determining a predisposition to infection, especially infection with HIV, together with therapy for the infection is described.

This invention relates to marker genes, and to uses thereof. More particularly, the present invention relates to marker genes which show a predisposition to the contraction of infection.

It is known in the art that it is possible to diagnose a predisposition to certain diseases with the use of marker genes. For example, oncogenes or tumour suppressor genes are widely regarded as being indicative of a susceptibility to certain cancers, especially in view of the associations between mutated oncogenes and deleted tumour suppressor genes and certain cancers. Additionally, genes have been identified, such as the BRCA genes, which are taken to be predictive of a greater risk of contracting cancers, for example breast cancers. It is also known that some individuals are highly susceptible or resistant to infection, especially viral infection. Prediction of disease susceptibility is beneficial for those possessing predisposing genes in order to avoid unnecessary contacts with known aetiological agents, chemicals, or viruses, and to take known and developing preventative means. It is also useful in the design of a vaccine against viral disease or for gene therapy.

Additionally, the development of an effective vaccine against major viral diseases such as human immunodeficiency virus (HIV) infection is a pressing matter with global socioeconomic ramifications. HIV is the causative agent of acquired immunodeficiency syndrome (AIDS). One of the keys to the development of such a vaccine is the understanding of the mechanisms of natural resistance against HIV infection. Several host genes have been associated with possible resistance against HIV infection and with either delayed or accelerated development of AIDS after HIV seroconversion [1]. These host genes include genes encoding chemokine receptors and cytokines, killer immunoglobulin-like receptors (KIRs) that serve as ligands for natural killer cell receptors, and those within the major histocompatibility complex (MHC) [1-11].

As an example of natural resistance against HIV infection, it is known that there are some individuals who, despite continued exposure to HIV, do hot become HIV positive. Some of the naturally resistant individuals possess a mutated HIV co-receptor gene known as CCRΔ32 [1-5]. However, this mutation is recessive and the homozygosity that confers resistance against HIV entry into cells is only rarely found. Thus, the above mutation cannot account for the majority of individuals who show spontaneous resistance against HIV infection. Among existing human clusters showing natural resistance against HIV infection, there is a distinct group of people known as HIV-exposed sero-negatives (ESNs) or HlV-1-exposed and uninfected individuals (EUIs) who have evidence of multiple and repeated exposure to HIV, but nevertheless possess no serum IgG antibodies reactive to HIV [12, 13]. Detection of HIV antigen-specific T-lymphocyte responses and of HIV-reactive IgA antibodies in urethral or vaginal secretions from these ESNs/EUIs show that they have been exposed to HIV but that the exposure has not resulted in infection [10-17]. Attempts to associate the ESN/EUI status with the previously reported genetic polymorphisms have so far been unsuccessful [10,14].

Additionally, the absence of clinical progression in some HIV-1-infected individuals and the lack of detectable HIV-1 genome despite multiple and repeated exposure to this virus in some apparently resistant groups of people are two notable phenomena when considering the development of preventative and therapeutic means to HIV infection [18-20]. Phenotypically, there are individuals who show strong HIV-1 antigen-specific T-lymphoryte responses and HIV-1-reactive mucosal. IgA production despite the absence of detectable plasma HIV-1 RNA and HIV-1 cDNA from peripheral blood mononuclear cells (PBMCs) [21-23]. They are often referred to as HIV-1-exposed and uninfected individuals (EUIs). Demonstration of HIV-1-neutralizing activity exerted by the mucosal IgA isolated from such EUIs [24-26] has suggested that rapid production and class switching of HIV-1-neutralizing antibodies might contribute to the presumable immune resistance against HIV infection. Protective roles of neutralizing antibodies against HIV-1-related simian immunodeficiency virus (SIV) or pathogenic chimeras between HIV-1 and SIV have also been demonstrated by passive transfer and vaccine-induced active immunization experiments in non-human primates [27-30]. However, genetic factors that may influence the effective production of possibly protective mucosal anti-HIV-1 antibodies are currently unknown.

However, by studying DNA samples from such individuals, with their informed consent, the present inventors have found that ESNs possess distinct rare alleles at microsatellite loci within a region of chromosome 22 that is syntenic to the area of mouse chromosome 15 containing a retrovirus resistance gene, Rfv-3. Thus, the present inventors have identified specific genotypes or polymorphisms which appear to be associated with resistance to HIV infection. This is the first demonstration that naturally acquired immune resistance against the establishment of HIV infection known as the ESN or EUI-status may be genetically influenced.

Hence, the present inventors have identified a marker gene, and polymorphisms thereof, which is indicative of a predisposition to infection, particularly viral infection and more particularly HIV infection.

Accordingly, the present invention provides a method of determining a predisposition to infection, the method comprising the steps of obtaining a DNA bearing sample from a subject, and assaying the sample to identify the alleles present at at least one of the microsatellite loci D22S929, D22S277, D22S264, D22S1166, D22S423, D22S1169, D22S418 or D22S272 wherein the presence of particular alleles at microsatellite loci D22S929, D22S277, D22S264, D22S1166, D22S423, D22S1169, D22S418 or D22S272 is indicative of a resistance to infection.

The present invention also encompasses nucleic acids complementary to these loci, including complementary RNA, together with the amino acid sequence encoded thereby and homologues, splice variants or functional equivalents thereof.

Preferably, the method is used for the determination of a predisposition to viral infection in an individual. It is preferred that the predisposition to a viral infection is for a virus such as a retrovirus, for example, an oncovirus, a lentivirus, or a spumavirus. HTLV and BLV (bovine leukaemia virus) are examples of oncoviruses which cause leukaemia. HIV and SIV are examples of lentiviruses which cause inflammatory and wasting disease. Human spumavirus is an example of a spumavirus. Most preferred is the determination of a predisposition to HIV infection.

The retrovirus may be an endogenous retrovirus, that is, a virus which is produced within the cell in response to certain physiological stimuli. Endogenous viruses are not spread by infection but are inherited. For example, a child born to an HIV-positive mother will generally be HIV positive, although seroconversion may take place after birth especially if the infant is not breast-fed.

Thus, the term “infection”, as used herein, is intended to include endogenous retroviruses and their activation, as well as the conventional sense of infection by exposure to an infectious agent. Human endogenous retroviruses (HERV) are examples of endogenous retroviruses.

The sample may be obtained invasively or non-invasively. Preferred samples include, blood; urine, semen, mouth swabs, skin cells, nail clippings, hair, or cervical smear samples.

Preferably, DNA isolated from the sample is amplified by the use of a nucleic acid amplification technique, such as PCR or rolling circle replication or other conventional nucleic acid amplification technique. Any nucleic acid amplification technique may be used with equal utility in the invention and it is not intended to limit the invention to the above described methods.

Accordingly, the invention also provides a method in which the sample is assayed for the presence or absence of particular genotypes at the microsatellite locus or loci using DNA fragment length analysis, DNA hybridisation techniques, DNA sequence identification, single strand length polymorphism (SSLP) analysis, or reference strand conformation (RSC) analysis.

More specifically, the present invention provides an assay which uses single strand length polymorphism (SSLP) analysis and the flanking primer set for PCR amplification of the microsatellite marker is selected from

(SEQ ID No:1)

D22S277 left, TTCTTGTGTGGTAGTCTGGG;

(SEQ ID No:2)

D22S277 right, TACCNACTCCCCAAACTATG;

(SEQ ID No:3)

D22S272 left, GAGTTTTGTTTGCCTGGCAC;

(SEQ ID No:4)

D22S272 right, AATGCACGACCCACCTAAAG;

(SEQ ID No:5)

D22S276 left, CATTCTGCCAAGCAATTTAT;

(SEQ ID No:6)

D22S276 right, GCTGCTCTTTAAGTTTCTTGACC;

(SEQ ID No:7)

D22S929 left, GGAGCTGCATGTACTAGCTGG;

(SEQ ID No:8)

D22S929 right, GCATTTATGGAGTATCCACAG;

(SEQ ID No:9)

D22S1169 left, GCACACACATGCACATAATC;
and

(SEQ ID No:10)

D22S1169 right, AACAACTTCCAGCAGACG.

complementary nucleic acids or fragments, polymorphisms, splice variants or homologues thereof.

The sample is preferably assayed for the presence or absence of particular genotypes at the indicated microsatellite loci using DNA fragment length analysis, DNA hybridisation techniques, DNA sequence identification, single strand length polymorphism (SSLP) analysis, or reference strand conformation (RSC) analysis which are well known in the art.

The sequence of the flanking primer sets for PCR amplification of the microsatellite markers is given in the examples below. Fragments, polymorphisms or homologues of these sequences are also included in the scope of the present invention.

In a second aspect, the invention also provides a kit for the diagnosis of a predisposition to infection, the kit comprising reagents for determination of genotype at at least one of the microsatellite loci D22S929, D22S277, D22S264, D22S1166, D22S423, D22S1169, D22S418 or D22S272

In a preferred embodiment the reagents are PCR primers for amplification of SSLP markers. The diagnostic kit can be used to categorize people into genetic groups among which the effects of prophylactic and therapeutic means for infection are compared and evaluated. Hence, unnecessary use of a therapeutic means on people who possess natural resistance can be avoided, and the dosage of therapeutic agents reduced, while the use of prophylactic means and vaccines can be concentrated on people such as those with extreme susceptibility in whom the effort can be expected to be more successful.

Hence, the present invention also encompasses the use of at least one of the microsatellite loci D22S929, D22S277, D22S264, D22S1166, D22S423, D22S1169, D22S418 or D22S272 in the determination of a predisposition to viral diseases, especially retroviral diseases and more particularly to HIV infection. Combinations of the loci may also be used.

It is important to note that it is the presence of the particular alleles which indicates a resistance to infection and that hence the absence of the particular alleles of the invention indicates a predisposition or susceptibility to contraction of the infection. In the most preferred embodiment of the invention, in the infection in question is the HIV virus the causative agent of AIDS. In this respect, the present invention provides a method of determining whether an individual is resistant to HIV infection or whether they are susceptible to it and are therefore highly at risk of contracting the disease.

Hence, the present invention also provides a method of determining a resistance to infection, the method comprising the steps of obtaining a DNA bearing sample from a subject, and assaying the sample to identify the alleles present at at least one of the microsatellite loci D22S929, D22S277, D22S264, D22S423 & D22S418 or D22S272, wherein the presence of particular alleles at microsatellite loci D22S929, D22S277, D22S264, D22S423 & D22S418 or D22S272 is indicative of a resistance to infection.

In another aspect the present invention provides a method for diagnosing a predisposition to HIV infection, the method including the step of assaying a DNA sample for the presence or absence of one, a plurality or each of the microsatellite loci D22S929, D22S277, D22S264, D22S1166, D22S423, D22S1169, D22S418 or D22S272.

In a further aspect, the invention also provides a method of treatment such as gene therapy to treat a subject having a predisposition to infection by the use of a vector bearing the chromosomal fragment that harbours at least one of the microsatellite loci D22S929, D22S277, D22S264, D22S1166, D22S423, D22S1169, D22S418 or D22S272 As described below, it is highly conceivable that a gene which influences immune cell functions associated with resistance against viral infection is located in the segment of human chromosome adjacent to the above microsatellite loci. Without wishing to be bound by theory, it is envisaged that this gene, putatively referred to as the human homologue of mouse Rfv-3, confers infection resistance to a subject individual, and hence it is a further aspect of the invention to use this gene, its transcripts, expressed peptide, polypeptide or protein, glycosylation, sulphonation, acetylation, or other post-translational derivatives, functional derivatives, homologues or fragments thereof in the treatment, prophylaxis or therapy of infection.

The microsatellite loci D22S929, D22S277, D22S264, D22S1166, D22S423, D22S1169, D22S418 or D22S272 all encode peptides, polypeptides or protein fragments. These peptides, polypeptides or protein fragments and their secondary or tertiary derivatives are all associated with viral resistance and their use in a person who has been identified as having a predisposition to viral infection may be beneficial in the prevention or prophylaxis of infection.

Additionally, the glycosylation, sulphonation, phosphorylation, acetylation or other addition or substitution products, homologues, splice variants, transcription variants or products derivable from the nucleic acid sequence of the microsatellite loci may used for this purpose and hence are considered to constitute part of the present invention.

Accordingly, the present invention also provides a composition comprising a peptide, polypeptide or protein fragment encoded by one or more of the microsatellite D22S929, D22S277, D22S264, D22S1166, D22S423, D22S1169, D22S418 or D22S272 The present invention also provides a pharmaceutical composition comprising a peptide, polypeptide or protein fragment encoded by one or more of the microsatellite loci D22S929, D22S277, D22S264, D22S1166, D22S423, D22S1169, D22S418 or D22S272 for use in the treatment or prophylaxis of infection.

The composition may be a microbicide, a pharmaceutical composition or a diet supplement or food additive. The pharmaceutical composition may comprise the additives generally known in the art of formulation chemistry such as vehicles, solvents, diluents, binders, excipients, anti-caking agents, preservatives, buffers, stabilisers, or humectants. The formulation may be in the form of tablets, emulsions, creams, suspensions, injectable suspensions, syrups, suppositories, pessaries, patches, impregnated implants or other conventional delivery method.

Where the composition is a microbicide, it is preferred that the composition is provided in a mucosally administerable formulation, for example for oral, nasal, rectal or vaginal application. For rectal or vaginal application, the microbicidal composition may be in the form of a gel, cream, suppository, pessary or other conventional form for rectal or vaginal application.

The microbicide may also be used for mucosal vaccination against viral disease, especially when the composition of the invention is used in a manner such as to provoke an immunological reaction.

Ideally, where the viral infection to be prevented is HIV which may be passed through sexual contact, the composition may be combined with a contraceptive. In such a case, the contraceptive may be a diaphragm, a cervical cap, a condom, a sponge or other intra-vaginal or barrier device, a coated IUD device, an oral contraceptive pill, a contraceptive implant or injection or a spermicidal gel, pessary, foam, film or cream.

Therefore, in a further aspect the present invention provides a contraceptive further comprising a comprising a peptide, polypeptide or protein fragment encoded by one or more of the microsatellite loci D22S929, D22S277, D22S264, D22S1166, D22S423, D22S1169, D22S418 or D22S272

The contraceptive of the invention may also further comprise the secondary or tertiary derivatives, glycosylation, sulphonation, phosphorylation, or other addition or substitution products, homologues, transcription variants or products derivable from the nucleic acid sequence of the microsatellite loci D22S929, D22S277, D22S264, D22S1166, D22S423, D22S1169, D22S418 or D22S272.

In a further aspect, the present invention also provides for the use of a peptide, polypeptide or protein fragment encoded by one or more of the microsatellite loci D22S929, D22S277, D22S264, D22S1166, D22S423, D22S1169, D22S418 or D22S272 in the preparation of a medicament for the treatment of infection.

Additionally, the present invention extends to the use of comprise the secondary or tertiary derivatives, glycosylation, sulphonation, phosphorylation, or other addition or substitution products, homologues, transcription variants or products derivable from the nucleic acid sequence of the microsatellite loci D22S929, D22S277, D22S264, D22S1166, D22S423, D22S1169, D22S418 or D22S272 in the preparation of a medicament for the treatment of infection.

Preferably, the infection is viral infection and more preferably retroviral infection. In the most preferred embodiment, the virus is the HIV or HTLV virus.

The invention also provides a method of determining a resistance to infection, the method comprising the steps of obtaining a DNA bearing sample from a subject, and assaying the sample to identify the alleles present at at least one of the microsatellite loci D22S929, D22S277, D22S264, D22S423 & D22S418 or D22S272, wherein the presence of particular alleles at microsatellite loci D22S929, D22S277, D22S264, D22S423 & D22S418 or D22S272 is indicative of a resistance to infection.

The methods of the present invention may be carried out using a kit containing the relevant microsatellite markers. Accordingly, the present invention also provides a kit for the diagnosis of a predisposition to infection, the kit comprising reagents for determination of genotype at at least one of the microsatellite loci D22S929, D22S277, D22S264, D22S423 & D22S418 or D22S272. More preferably, the kit comprises reagents for determination of genotype for at least one of the microsatellite loci D22S929, D22S277, D22S264, D22S423 & D22S418 or D22S272 or a nucleic acid complementary thereto, fragments, polymorphisms, splice variants or homologues thereof.

In another aspect of the invention, the DNA of the microsatellite loci may be incorporated into a vector. For research purposes, that vector may be used to transfect cells. The vector may also be used in the preparation of a medicament, for example for the treatment of infection or in the formation or formulation of a DNA vaccine, or in gene therapy.

Hence, in another aspect, the present invention also provides a vector bearing the chromosomal fragment which harbours at least one of the microsatellite loci D22S929, D22S277, D22S264, D22S423 & D22S418 or D22S272 or a nucleic acid complementary thereto, or fragments, polymorphisms, splice variants or homologues thereof.

Use of such a vector in medicine would be beneficial in the prophylaxis or treatment of infection, particularly viral infection and especially for infection by the HIV virus.

The DNA of these microsatellite loci is particularly useful in the production of a DNA vaccine. A DNA vaccine of the invention may comprise naked, adjuvantised or encapsulated DNA. The DNA sequences of the loci may be used in the preparation of a vaccine which uses nucleic acid sequences complementary to the DNA sequences of the loci, or fragments, polymorphisms, splice variants or homologues thereof.

Such a vaccine may be orally or parenterally administerable. Examples of parenteral administration include by injection, whether subcutaneous, intravenous or intramuscular, by inhalation and mucosal administration. It is not, however, intended that these examples of parenteral administration are limited or exhaustive.

The vaccine of the present invention is preferably immunogenic.

For the preparation of vaccines or other pharmaceuticals, it is useful to identify compounds which bind to, or otherwise recognise, and/or modify or modulate the DNA encoding these loci, together with their fragments, polymorphisms, splice variants, complementary nucleic acids or homologues. It is useful if these compounds are provided in the form of a chip or assay plate, especially where many compounds are to be screened. Hence, the present invention also provides for the use of DNA encoding the microsatellite loci D22S929, D22S277, D22S264, D22S423 & D22S418 or D22S272, or fragments, polymorphisms, splice variants, complementary nucleic acids or homologues of said gene in chip or assay plate for screening of compounds able to bind to or otherwise recognise said DNA.

In this respect, the invention also provides a chip or assay plate comprising DNA encoding the microsatellite loci D22S929, D22S277, D22S264, D22S423 & D22S418 or D22S272, or fragments, polymorphisms, splice variants, complementary nucleic acids or homologues of said gene.

The chip or assay plate may be used in medical diagnosis or research.

Since the DNA encodes an amino acid sequence which may also be used, the present invention also provides a chip or assay plate comprising a peptide, polypeptide, protein or glycosylation, sulphonation, acetylation, or other post-translational derivatives, functional derivatives, homologues or fragments of the protein encoded by the gene located in the chromosomal segment adjacent to the microsatellite loci D22S929, D22S277, D22S264, D22S423 & D22S418 or D22S272, complementary nucleic acids or fragments, polymorphisms, splice variants or homologues of said gene.

Additionally, the invention encompasses the use of such a chip or assay plate in screening of compounds able to bind to or otherwise recognise, to modify or mimic said peptide, polypeptide, protein or glycosylation, sulphonation, acetylation, or other post-translational derivatives, functional derivatives, homologues or fragments of the protein encoded by the gene located in the chromosomal segment adjacent to the microsatellite loci D22S929, D22S277, D22S264, D22S423 & D22S418 or D22S272, complementary nucleic acids or fragments, polymorphisms, splice variants or homologues of said gene.

The present inventors predict that the peptide, polypeptide or protein, and the nucleic acid sequence of the invention, stimulate the production of immunoglobulin A (IgA), which promotes a resistance to infection. Particularly, such compounds can be used to stimulate mucosal production of IgA, especially virus reactive mucosal IgA.

Hence, the present invention also provides the use of a peptide, polypeptide, protein or glycosylation, sulphonation, acetylation, or other post-translational derivatives, functional derivatives, homologues or fragments of the protein encoded by the gene located in the chromosomal segment adjacent to the microsatellite loci D22S929, D22S277, D22S264, D22S423 & D22S418 or D22S272, complementary nucleic acids or fragments, polymorphisms, splice variants or homologues of said gene to produce an immunoglobulin A which provides resistance to infection or possesses antiviral activity.

Additionally, the invention provides the use of the microsatellite loci D22S929, D22S277, D22S264, D22S423 & D22S418 or D22S272, complementary nucleic acids or fragments, polymorphisms, splice variants or homologues of said gene to trigger production of an immunoglobulin A which provides resistance to infection or possesses antiviral activity.

An IgA so produced can be used in a composition for the treatment or prophylaxis of infection, especially viral infection and most particularly HIV infection.

The IgA may be used alone or in combination with any of the above mentioned pharmaceutical compositions.

Host genetic factors influencing viral entry and replication and immune responses against retroviral infections have been extensively studied by using mouse models [31-34]. Friend mouse leukaemia virus complex (FV) is composed of replication-competent Friend mouse helper virus (F-MuLV) and defective spleen focus-forming virus. FV induces rapid proliferation of infected erythroid progenitor cells upon inoculation into immunocompetent adult mice of susceptible strains. Persistent infection of FV associated with severe immunosuppression ultimately causes the emergence of mono- or pligoclonal expansion of leukaemia cells due to an insertional activation of a cellular transcription factor or disruption of a tumour suppressor gene. Host gene loci, Fv-1-Fv-4, that directly control the viral entry and replication in the target cells have been identified [35-38]. However, even when the host animals share the same susceptible genotypes at the above loci, the rate of disease development and progression still changes drastically depending on host genotypes at several loci that influence immune responses to FV antigens [33]. Two major histocompatibility complex (MHC) class II loci directly restrict the T helper cell recognition of the viral envelope antigen [39, 40], while a class I locus influences the production of cytokines from viral antigen-specific T-cells [41]. Another locus mapped in the MHC class Ib region may affect natural killer cell functions [42, 43]. Yet another host locus that has been mapped in chromosome 15, and thus is irrelevant to MHC, strongly influences the persistence of viraemia after FV infection [33, 44-46]. Genotypes at the same non-MHC locus also influence the production of cytotoxic antibodies that modulate the expression of viral antigens on infected cell surfaces [47]. However, possible relationship between the persistence of viraemia and production of virus-neutralizing antibodies has not been directly examined. Here we have performed linkage analyses on a mouse locus that influences the production of virus-neutralizing antibodies upon FV infection.

An extension of this mouse study unexpectedly led the present inventors to a demonstration of human chromosomal markers that are associated with strong immune responses to HIV-1 in HIV-uninfected individuals.

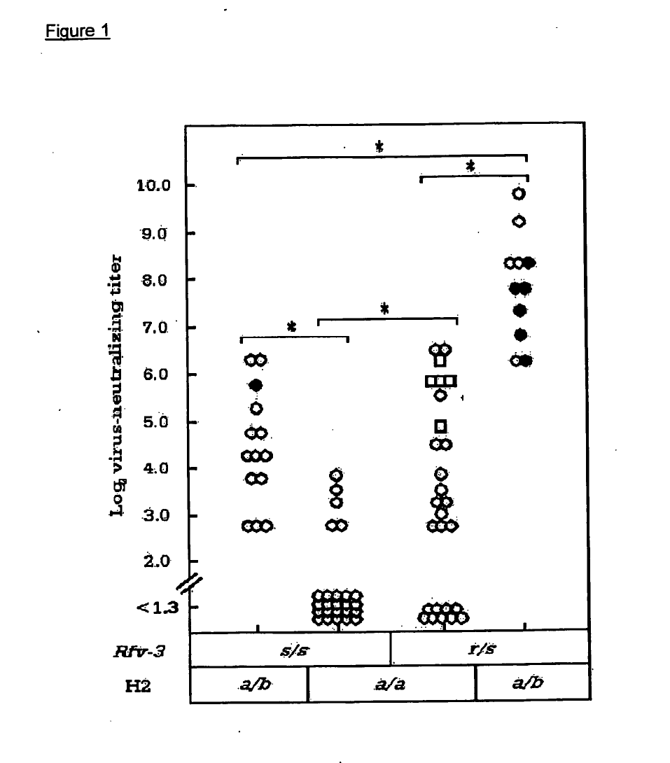

The gene Rfv-3 was originally defined as a single autosomal gene that determines whether mice infected with Friend leukaemia retrovirus recovered from viraemia by 30 to 60 days after infection or not [49, 50]. This gene has been mapped to mouse chromosome 15 [51, 52], although its molecular identity is still unknown. Immune resistance against Friend retrovirus infection is also influenced by genes of mouse major histocompatibility complex (MHC), H2, which control T-lymphocyte responses to the viral envelope antigen [20, 53, 54]. When tested in congenic strains, early production of virus-neutralizing antibodies was observed in mice that possessed either a resistant allele (Rfv-3^{r}) at the Rfv-3 locus or a responder haplotype (H2^{b}) at mouse MHC (FIG. 1), suggesting that Rfv-3 and H2 may effect the immune system through a common pathway. Moreover, mice possessing both an Rfv-3 allele and an H2^{b} haplotype showed even higher levels of virus-neutralizing antibodies and a higher frequency of IgM to IgG class switching in comparison with the H2^{a/b} mice lacking an Rfv-3^{r} (FIG. 1), further indicating that Rfv-3, in cooperation with H2, might regulate a T-helper cell function. This was of potential relevance to why HIV-specific IgA production, in the apparent absence of IgG, can be detected in ESNs (see Table 1 for examples), especially because HIV-1 antigen-specific T helper cell responsiveness and patterns of cytokine production from T cells may differ between ESN and HIV-infected individuals [14, 16, 48].

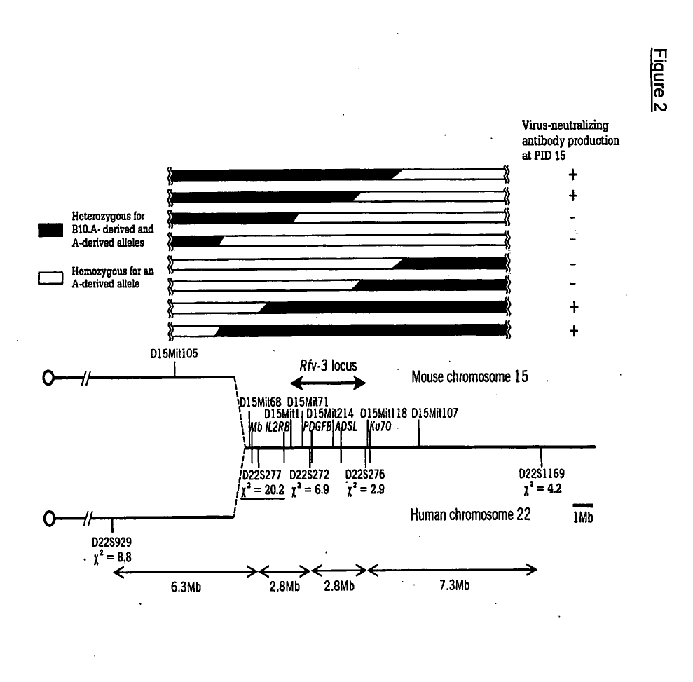

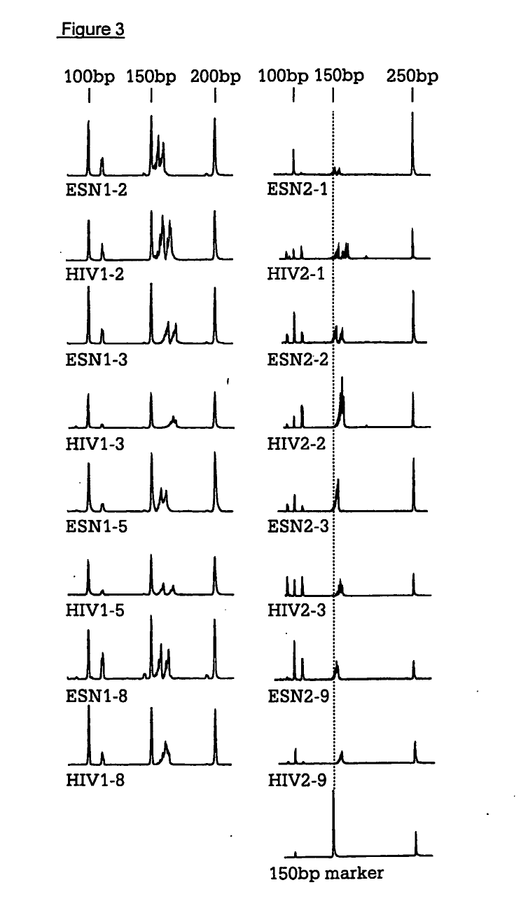

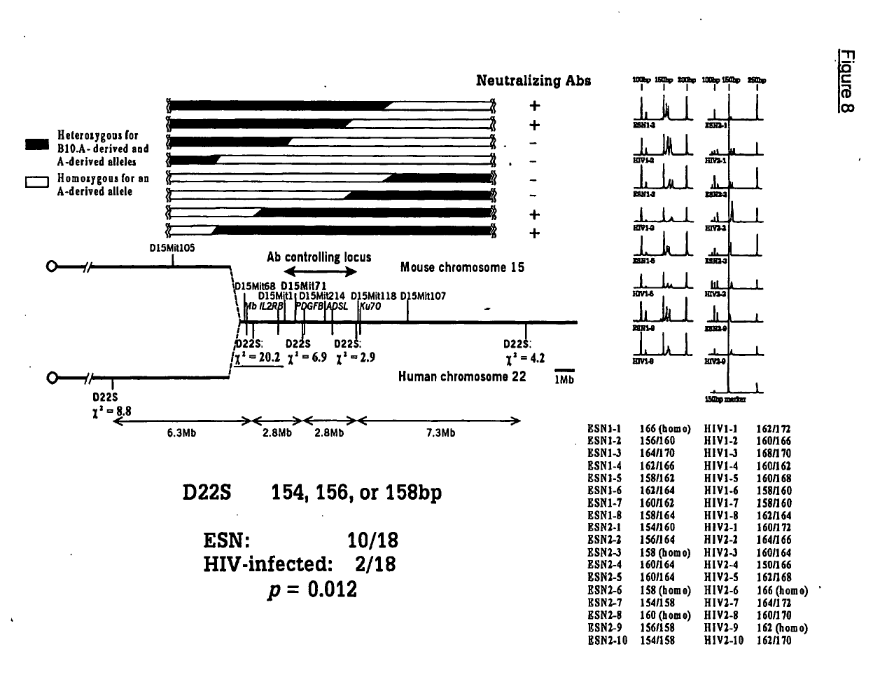

Using (B10.A×A)×A backcross mice in which Rfv-3-associated phenotypes defined by titres of virus-neutralizing antibodies at post-infection day (PID) 15 were segregated, the present inventors performed detailed linkage analyses, and mapped this gene within a 3-Mbp segment of mouse chromosome 15 between D15Mit1 and D15Mit118 (FIGS. 2, 4, 5, 8). For physical mapping of the mouse markers, information found in the Ensemble Genome Browser (http://www.ensembl.orq) on the segment of human chromosome 22 that is syntenic to this region of mouse chromosome 15 was utilized, and genes homologous between the two species were lined up with known simple sequence length polymorphism (SSLP) markers. As a result, several SSLP loci that are both polymorphic and located in the vicinity of the human homologues of Rfv-3-linked genes were identified (FIGS. 2, 4, 5, 8). Peripheral blood mononuclear cells (PBMCs) were provided by the previously documented ESN and HIV-infected individuals under written informed consent. All the ESNs enrolled were tested for serum HIV-1-reactive IgG, plasma HIV RNA, and HIV cDNA in cells of seminal or vaginal secretions, and none of them showed the presence of HIV-1, while the HIV-infected individuals were all positive in these tests. Using genomic DNA isolated from the above PBMCs as templates, alleles at the SSLP loci within chromosome 22 were identified by determining the sizes of PCR-amplified fragments (FIG. 3 and Table 1). As a result, the distribution of allele frequencies at the D22S277 locus was significantly different (X²=20.2 for the 2×11 table, p=0.020 by Fisher's exact test) between the ESN and HIV-infected groups. Viewed differently, ten of the 18 ESNs tested possessed at least one of the three distinct alleles at the D22S277 locus yielding a 154-, 156-, or 158-bp fragment, while one of these alleles were found in only two individuals among the 18 HIV-infected persons tested (X²=8.0, p=0.012 by Fisher's exact test). These three alleles are among the rare ones with reported frequencies of 7, 5, and 9%, respectively, in The Genome Database (htt):/tadbwww.qdb.orog/). On the other hand, the alleles yielding a 160- or 162-bp fragment are rather common among Caucasians with reported frequencies of 29 and 14%, respectively, and these latter alleles were observed at comparable frequencies in both groups (Table 1). Therefore, it should be emphasized that two of the 18 ESNs were homozygous for the allele yielding a 158-bp fragment, and three were zygotes between two of the above three rare alleles. In contrast, such zygotes of the rare alleles were not found among the HIV-infected individuals (5 of 18 vs 0 of 18 yielding p=0.045 by Fisher's exact test).

The above accumulation of the distinct alleles at the D22S277 locus among the ESNs seems not coincidental, since less prominent but similar skewing in the distribution of distinct rare alleles was also observed at the surrounding chromosomal loci (Table 1). In fact, at D22S929 alleles yielding a 144- or 146-bp fragment were found in 5 among the 18 ESNs and one individual was even homozygous for one of the two alleles, while only one individual possessed such an allele among the 17 HIV-infected individuals tested. Rare alleles yielding either one of 132, 142, or 148-bp fragments were found in 5 of the 18 ESNs but in only one of the 18 HIV-infected individual tested at the D22S272 locus. However, neither the distribution of allele frequencies (FIG. 2, 4, 5, 8) nor the frequencies of individuals possessing a rare allele (Table 1) were significantly different between the ESN and HIV-infected groups at the loci apart from this region, such as D22S1169.

TABLE 1
Genotypes at SSLP loci within human chromosome 22 of HIV-
exposed seronegative and HIV-infected individuals.

| | HIV-1-reactive | | | | | | |
|---|---|---|---|---|---|---|---|
| | antibodies in urethral | | | | | | |
| | or vaginal swabs | | | | | | |
| | (ELISA absorbance) | | SSLP loci | | | | |
| Individuals | IgA | IgG | D22S929 | D22S277 | D22S272 | D22S276 | D22S1169 |
| ESN1-1 | 0.784 | 0.002 | 132/142 | 166 | 134/140 | 243/251 | 126/128 |
| | | | | (homo) | | | |
| ESN1-2 | 0.811 | ND | 140 | 156/160 | 134 | 245 | 120/126 |
| | | | (homo) | | (homo) | (homo) | |
| ESN1-3 | 0.217 | ND | 138/144 | 164/170 | 134 | 245 | 120/126 |
| | | | | | (homo) | (homo) | |
| ESN1-4 | 0.557 | 0.021 | 144 | 162/166 | 132/148 | 243/249 | 126/130 |
| | | | (homo) | | | | |
| ESN1-5 | 0.911 | 0.006 | 138 | 158/162 | 132/142 | 243/251 | 126 |
| | | | (homo) | | | | (homo) |
| ESN1-6 | 0.217 | 0.006 | 138/140 | 162/164 | 134/142 | 245/251 | 128/130 |
| ESN1-7 | 0.248 | 0.001 | 138/146 | 160/162 | 134/136 | 243/251 | 126/128 |
| ESN1-8 | 0.852 | 0.004 | 138/140 | 158/164 | 132/140 | 243/245 | 126/128 |
| ESN2-1 | 1.012 | ND | 134/144 | 154/160 | 140/150 | 241/253 | 120/126 |
| ESN2-2 | 0.478 | ND | 134/142 | 156/164 | 134/140 | 243 | 118/126 |
| | | | | | | (homo) | |
| ESN2-3 | 0.263 | 0.008 | 134/136 | 158 | 134/148 | 241/243 | 120/128 |
| | | | | (homo) | | | |
| ESN2-4 | 0.171 | 0.008 | 138/146 | 160/164 | 134/140 | 243/245 | 126/130 |
| ESN2-5 | 0.279 | 0.007 | 138/140 | 160/164 | 134/136 | 243/245 | 118/126 |
| ESN2-6 | 0.246 | 0.008 | 138 | 158 | 134/140 | ND | 120 |
| | | | (homo) | (homo) | | | (homo) |
| ESN2-7 | 0.204 | 0.008 | 140/142 | 154/158 | 134 | ND | 120/130 |
| | | | | | (homo) | | |
| ESN2-8 | 0.200 | 0.008 | 138 | 160 | 134 | ND | 126/128 |
| | | | (homo) | (homo) | (homo) | | |
| ESN2-9 | 0.206 | 0.006 | 138 | 156/158 | 140 | 243 | 118/128 |
| | | | (homo) | | (homo) | (homo) | |
| ESN2-10 | 0.101 | 0.008 | 132/140 | 154/158 | 134 | 241 | 120/126 |
| | | | | | (homo) | (homo) | |
| HIV1-1 | 0.114 | 1.112 | 138 | 162/172 | 140 | 243/251 | 126/130 |
| | | | (homo) | | (homo) | | |
| HIV1-2 | 0.300 | 1.080 | 138/140 | 160/166 | 134 | 243/245 | 120/126 |
| | | | | | (homo) | | |
| HIV1-3 | 0.201 | 0.754 | 134/138 | 168/170 | 134/136 | 243 | 126 |
| | | | | | | (homo) | (homo) |
| HIV1-4 | 0.308 | ND | 132/138 | 160/162 | 134/140 | 243/245 | 126 |
| | | | | | | | (homo) |
| HIV1-5 | 0.121 | 0.587 | 134/138 | 160/168 | 134 | 243/245 | ND |
| | | | | | (homo) | | |
| HIV1-6 | 0.114 | 1.120 | 132/136 | 158/160 | 134/140 | 243/251 | 118/130 |
| HIV1-7 | 0.201 | 0.880 | 136/140 | 158/160 | 134/136 | 241/243 | 126/130 |
| HIV1-8 | 0.412 | 0.951 | 136/138 | 162/164 | 134/150 | 245 | 118/126 |
| | | | | | | (homo) | |
| HIV2-1 | 0.408 | 0.741 | 138/144 | 160/172 | 134/140 | ND | 128/130 |
| HIV2-2 | 0.301 | 0.983 | 138 | 164/166 | 134/140 | ND | 126 |
| | | | (homo) | | | | (homo) |
| HIV2-3 | ND | ND | 140/142 | 160/164 | 134/142 | ND | 118/126 |
| HIV2-4 | 0.303 | 0.360 | 136 | 150/166 | 134/140 | ND | 128/130 |
| | | | (homo) | | | | |
| HIV2-5 | 0.251 | 0.449 | 138/140 | 162/168 | 134/140 | 245 | 124/126 |
| | | | | | | (homo) | |
| HIV2-6 | 0.513 | 0.338 | ND | 166 | 134 | ND | 120 |
| | | | | (homo) | (homo) | | (homo) |
| HIV2-7 | 0.514 | 0.107 | 132/138 | 164/172 | 134 | ND | 126/130 |
| | | | | | (homo) | | |
| HIV2-8 | ND | ND | 138/142 | 160/170 | 140/144 | ND | 120 |
| | | | | | | | (homo) |
| HIV2-9 | 0.204 | ND | 138/142 | 162 | 134/136 | ND | 118/126 |
| | | | | (homo) | | | |
| HIV2-10 | 0.228 | ND | 138 | 162/170 | 134/140 | 245/251 | 128/130 |
| | | | (homo) | | | | |

Rare alleles (those with a reported frequency of less than 10%) at each locus are shown underlined in boldface.
ND, not determined;
homo, homozygous.
No serum IgG reactive to HIV-1 was detectable in the ESNs.

The above data are consistent with the hypothesis that a dominant responder allele of a gene, possibly homologous to mouse Rfv-3^{r}, that confers the ability to produce and class-switch anti-retroviral antibodies at an early stage after HIV exposure is present and located near the D22S277 locus, so that the ESN status co-segregates with the D22S277 alleles yielding a 154-, 156-, or 158-bp fragment. None of the previously reported human genes that affect the resistance against HIV-infection and/or the course of AIDS progression are located in this area of the human chromosome, as CCR5 and CCR2 being located at 3p21, SDF1 at 10q11.1, HLA at 6p21.3, KIRs at 19q13.4, and IL10at 1q31-32.

A well-established genetic basis for resistance against HIV infection is the homozygosity for a mutant form of the HIV co-receptor gene, CCR5, which results in the lack of cell surface expression of this receptor [1-5]. However, since the mutation, CCR5 Δ32, is rare and the homozygosity is found in only 1% of Caucasians [1, 3], it cannot account for the more common phenomenon of ESN status. In fact, the CCR5 Δ32 mutation was not found in the previously documented ESNs [10, 14, 48]. On the other hand, the majority of ESNs showed a higher IL-2 and lower IL-10 production from PBMCs in comparison with HIV-seropositives upon stimulation with HIV envelope-derived antigenic peptides [14]. Therefore, it is possible that some genes that regulate T-cell functions might differ between ESNs and HIV-seropositives. Since Rfv-3 seems to modulate some T-helper cell functions in mice (FIG. 1), identification of its molecular nature along with the analysis of its human homologue might provide us with an entirely new direction into the preventative and therapeutic means for HIV infection.

Embodiments of the invention will now be described, by way of example only, with reference to the following examples as illustrated by the appended drawings of which:

FIG. 1 shows titres of Friend virus-neutralizing antibodies detected at PID 16-20 in congenic strains of mice;

FIG. 2 is a diagrammatic presentation of the order of and distance between SSLP markers and homologous genes located within the syntenic region of mouse chromosome 15 and human chromosome 22;

FIG. 3 shows genotyping at the D22S277 locus;

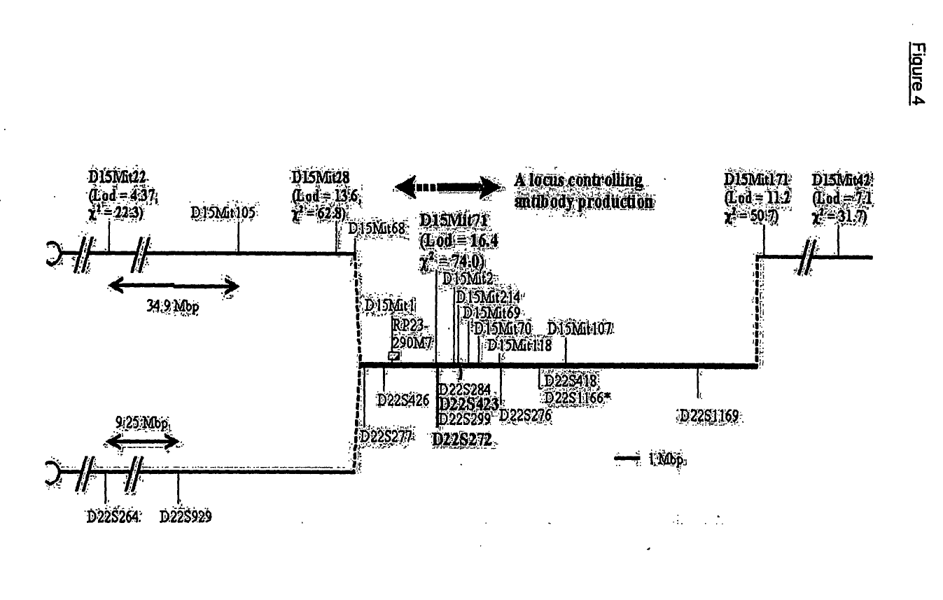

FIG. 4 shows the order of and distance between SSLP markers located within the syntenic region of mouse chromosome 15 and human chromosome 22; a locus controlling the anti-HIV IgA production located exactly syntenic to the mouse gene controlling anti-retroviral antibody production.

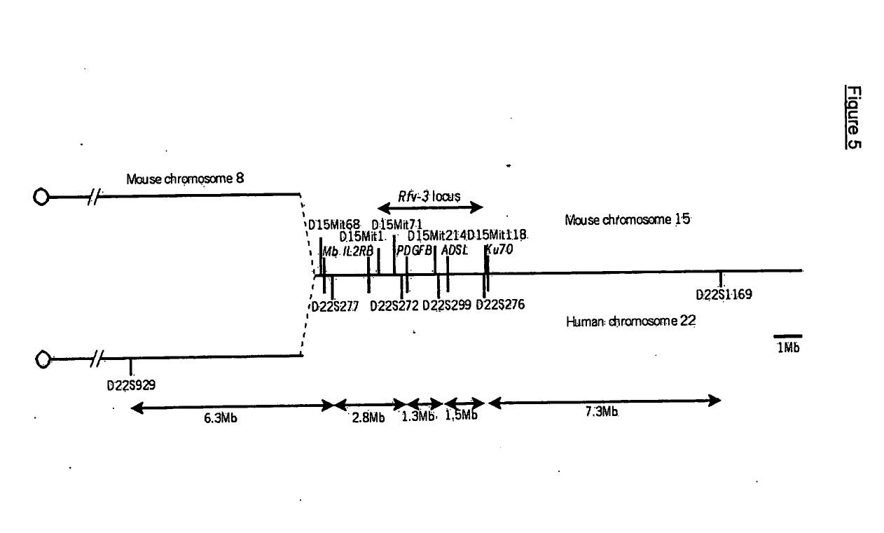

FIG. 5 shows the perfect identity between the mouse gene which confers resistance to Friend's virus and the human virus resistance gene;

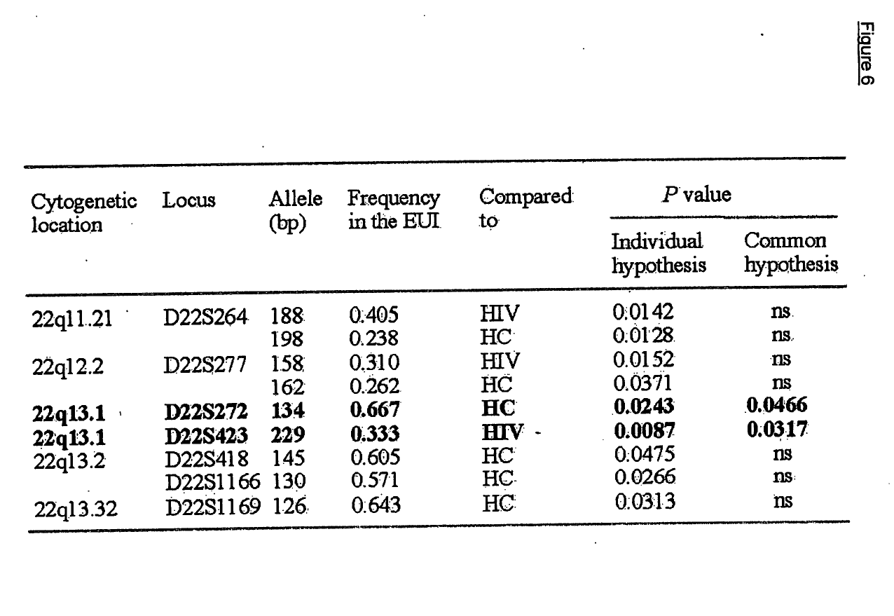

FIG. 6 is a diagrammatic representation showing the interruption of linkage disequilibrium across chromosome 22 observed only in the HlV-1-exposed but uninfected individuals which shows evidence of a past mutational or recombinational event which occurred in the ancestors of the HIV-1 exposed but uninfected indvidiuals; and

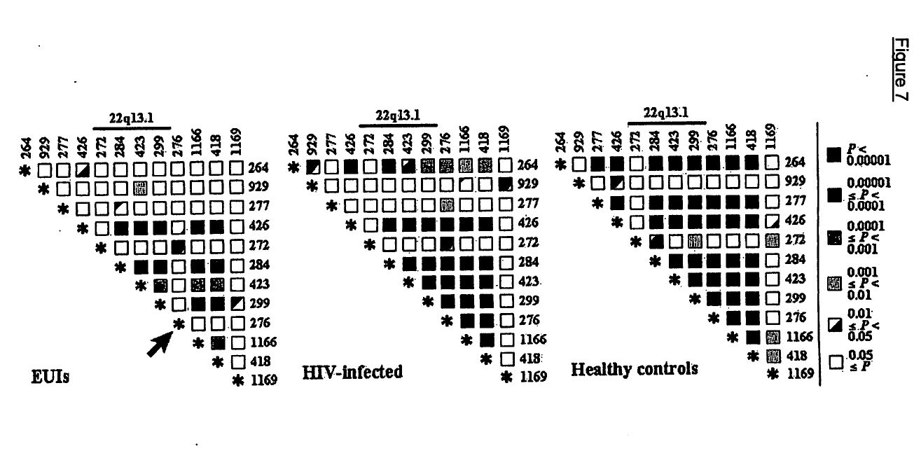

FIG. 7 shows site mapping and frequencies of HIV-1 resistant alleles together with diagrammatic representations of mouse antibody controlling locus on mouse chromosome 15 and syntenic regions of human chromosome 22, also shows interruption of linkage disequilibrium across chromosome 22 observed only in the HIV-1-exposed but uninfected individuals and evidence of a past mutational or recombinational event occurred in the ancestors of the HIV-1 exposed but uninfected individuals.

### EXAMPLES

### Example 1

As can be seen in FIG. 1, Mice were inoculated with either 150 spleen focus-forming units (SFFU) for H2^{a/a} or 1,500 SFFU for H2^{a/b} group of Friend retrovirus complex as described [54, 56, 57]. They were bled from the retro-orbital sinus under ether anaesthesia at PID 16-20, and each serum was tested for the ability to neutralize Friend leukaemia helper virus infectivity by focal immunoenzymatic assays [54, 56, 57]. Neutralizing titres of the whole serum obtained from each individual mouse are shown. Sera from H2^{a/b} mice were also tested for the presence of IgG class of neutralizing antibodies by treating them with 50 mM 2-mercaptoethanol as described [54, 56, 57], and those that contained virus-neutralizing IgG are shown with closed symbols. H2a/a (B10.A×A)×A backcross mice were divided into either Rfv-3^{r} or Rfv-3^{r/s} group based on their experimentally identified D15Mit71 alleles. Antibody titres of (B10.A×A)F₁ mice that are H2^{a/a} and Rfv-3^{r/s} by definition are shown with square symbols. Mean titres were compared between each genetic group by Student's t test, and asterisks indicate a statistically significant difference (p<0.0001). Frequency of individuals (6/11) that possessed neutralizing IgG among the Rfv-3^{r/s}, H2^{a/b} mice is significantly higher than that (1/14) of the Rfv-3^{s/s}, H2^{a/b} mice (p=0.02 by Fisher's exact test).

In the diagrammatic representation of FIGS. 2, 4 and 5 centromeres (O) are placed on the left. The location of the Rfv-3 locus was determined by linkage analyses using 185 (B10.A×A)×A backcross mice. The strongest linkage (X²=62.2) between SLLP genotypes and virus-neutralizing antibody titres at PID 15 was observed at the D15Mit71 locus. The location of the Rfv-3 locus was further narrowed to the shown area by correlating the SSLP genotypes and antibody titres in the 8 critical animals in which a chromosomal recombination was identified between the D15Mit105 and D15Mit107 loci. Difference in the distribution of allele frequencies at each human locus between the ESN and HIV-infected groups was analysed by performing a X² analysis for a 2× (number of alleles) table. The above-determined X² value is shown below the name of each microsatellite locus.

FIG. 3 shows genotyping at the D22S277 locus where PCR and fragment analyses were performed at least three times for each sample, and representative results from two separate experiments are shown herein.

ESN and HIV-serooositive Individuals

Eighteen heterosexual couples discordant for HIV serostatus were enrolled in the study. In 11 couples the female partner was HIV-infected, whereas the male partner was HIV-seronegative despite a prolonged history of penetrative sexual intercourse without condom. In the remaining 7 couples the male partner was HlV-infected, whereas the female partner was HIV-seronegative. Inclusion criteria for the ESN were a history of multiple unprotected sexual episodes for at least 4 years with at least an episode of at-risk intercourse within the 4 months prior to the study period. The couples reported an average of 8 unprotected sexual episodes/year (range 5 to >40) in the 4 years. Vaginal sex was the rule, and oral sex was only very rarely practiced. Anal sex was not reported by any couple. HIV-1 viraemia was tested as described previously [14, 16], and was undetectable in all the ESNs. To exclude the possibility of a mucosally-confined presence of HIV-1 in the ESNs, cDNA was analyzed in seminal or vaginal fluid as described [14, 16]. HIV-1 cDNA was detected in all the HIV-infected individuals but not in the ESNs. Titration of HIV-1-specific antibodies in serum and urethral or vaginal swabs was performed as described [14-17, 48, 55]. The ESNs possessed no detectable HIV-reactive IgG in their serum, while all the HIV-infected individuals were positive for serum IgG reactive to HIV-1. All the analyses were performed in a blinded fashion. The Research Ethics Committees of the Luigi Sacco Hospital, Milano, and of the Santa Maria Annunziata Hospital, Florence, have approved this protocol. Written informed consent was obtained from all patients before the enrollment.

Chromosomal Mapping of the Rfv-3 Gene

(B10.A×A)×A backcross mice were bred, infected with 150 spleen focus-forming units (SFFU) of Friend virus complex, and bled from the retro-orbital sinus at PID 15 as described [56, 57]. Serum titres of Friend virus-neutralizing antibodies were determined as described [54, 56, 57]. Tail tips were cut to prepare genomic DNA, and alleles at SSLP loci D15Mit22, D15Mit28, D15Mit71, D15Mit171, and D15Mit42 were identified using specific pairs of PCR primers [51, 52]. Individuals that possessed a recombination between D15Mit 28 and D15Mit 171 were further analysed for their genotypes at the additional SSLP loci (FIGS. 2, 4, 5).

Analyses of Human SSLP Markers

Genomic DNA (0.5 μg) extracted from PBMCs of each ESN and HIV-infected individual was used as the template for 40 cycles of PCR amplification using the following flanking primer sets [58]:

(SEQ ID No:1)

D22S277 left, TTCTTGTGTGGTAGTCTGGG;

(SEQ ID No:2)

D22S277 right, TACCNACTCCCCAAACTATG;

(SEQ ID No:3)

D22S272 left, GAGTTTTGTTTGCCTGGCAC;

(SEQ ID No:4)

D22S272 right, AATGCACGACCCACCTAAAG;

(SEQ ID No:5)

D22S276 left, CATTCTGCCAAGCAATTTAT;

(SEQ ID No:6)

D22S276 right, GCTGCTCTTTAAGTTTCTTGACC;

(SEQ ID No:7)

D22S929 left, GGAGCTGCATGTACTAGCTGG;

(SEQ ID No:8)

D225929 right, GCATTTATGGAGTATCCACAG;

(SEQ ID No:9)

D22S1169 left, GCACACACATGCACATAATC;
and

(SEQ ID No:10)

D22S1169 right, AACAACTTCCAGCAGACG.

Left primer of each above set was labelled with Cy5 at 5′ end for detection in fragment analyses with a Long Read Tower DNA sequencer (Amersham Pharmacia Biotech UK, Ltd., Buckinghamshire, UK). PCR amplification was performed with recombinant Taq polymerase (Invitrogen Life Technologies, Carlsbad, Calif., U.S.A.) in the following conditions: initial denaturation at 94° C. for 2 min., each amplification cycle comprising 94° C. for 30 sec., 55° C. for 30 sec and 74° C. for 90 sec, with final elongation at 74° C. for 10 min. For genotyping each PCR amplified fragment (50-100 fmol) was applied to the above DNA sequencer along with appropriate size markers, and the fragment size was identified with the ALFexpress Sizer program according to the manufacturer's instruction.

### Example 2

Methods.

Mice and virus. Breeding pairs of B10.A/SIc and A/WySnJ mice were purchased from Japan SLC, Inc., Hamamatsu, Japan and The Jackson Laboratory, Bar Harbor, Me., respectively. These parental strains and (B1.A×A)F₁ and (B10A×A)×A backcross mice were bred and maintained in animal facilities at Rakuno Gakuen University and Kinki University School of Medicine under a specific pathogen-free condition. The following experimental procedures were approved by the animal experiment committee and performed under relevant guidelines of each university. Preparation of FV and intravenous inoculation were performed as described previously [42, 43, 64, 65].

Assays for Virus-Neutralizing Antibodies.

Mice were infected with 150 spleen focus-forming units of FV, and were bled from the retro-orbital sinus under ether anaesthesia on the indicated days after infection. Sera were collected and stored frozen until used. Serum titres of F-MuLV-neutralizing antibodies were determined as described previously [43, 64, 65]. In brief, serial two-fold dilutions of each serum was mixed with a standard dilution of a pooled supernate collected from cultures of a Mus dunni cell clone chronically infected with an infectious molecular clone of F-MuLV, FB29, incubated, and inoculated onto cultures of uninfected Mus dunni cells in 24-well tissue culture plates. Two days later, foci of F-MuLV-infected cells were visualized by a focal immunoenzymatic assay using a monoclonal antibody specific for the F-MuLV env gene product [74]. All assays were performed in duplicate wells for each serum dilution. Neutralization was judged to be significant when average number of infected cell foci was reduced to <¼ in comparison with that in the control wells where the virus was mixed with the diluent alone. Antibody titres were defined by the highest serum dilution that gave significant neutralization.

Analyses of Simple Sequence Length Polymorphisms (SSLP) in Mice.

Genomic DNA was prepared from the tail tip of each mouse using DNeasy Tissue Kit (QIAGEN GmbH, Hilden, Germany) according to the manufacturer's instructions. A pair of oligonucleotide primers for each microsatellite locus was prepared based on the sequence information listed in a database within the Genetic and Physical Maps of the Mouse Genome site (http://www-genome.wi.mit.edu/cgi-bin/mouse/, The Whitehead Institute/MIT Center for Genome Research, Massachusetts), and were used for amplification by polymerase chain reaction (PCR) of genomic DNA fragments. 50 ng of each template DNA was subjected to 35 cycles of amplification with Quick Thermo Personal PCR Systems (Nippon Genetics, Tokyo, Japan), using a recombinant Taq polymerase (Invitrogen Life Technologies, Carlsbad, Calif.) according to the manufacturer's instructions. PCR products were separated by electrophoresis in 4% agarose gel and were visualized under a UV light by ethidium bromide staining.

Linkage Analyses.

Backcross mice which possessed a significant titre (2^{1.5}≦) of F-MuLV-neutralizing antibodies were designated as producers, while those with an antibody titre below the limit of detection (<2^{1.3}) were designated as non-producers. The cut-off point was determined based on F-MuLV-neutralizing titres of 16 A/WySn mice (all were below the limit of detection) and the lowest neutralizing titre observed in a group of 15 (B10.A×A/WVySn)F₁ mice (2^{1.5}) at PID 15. Correlation between genotypes at each examined chromosomal locus and the presence or absence of virus-neutralizing antibodies was analyzed by Pearson's X² test on a contingency table of 2×2. Map orders of the chromosomal loci and Loci scores were determined by multipoint analyses using MAPMAKER/EXP software version 3.0b (The Whitehead Institute/MIT Center for Genome Research).

Physical Mapping of the D15Mit1 Locus.

38 overlapping clones of bacterial artificial chromosome (BAC) covering the segment of mouse chromosome 15 between the D15Mit68 and D15Mit118 loci were selected and obtained from the RPCI-23 female C57BL/6 mouse BAC library (Children's Hospital Oakland Research Institute, Oakland, Calif.). The presence of each microsatellite locus was detected by PCR assays as described above using each isolated BAC clone as a template. A PCR product of the expected size was obtained by using the primer set for D15Mit1 and RP23-290M7 DNA as a template, and the presence of the flanking primer and repeat sequences that match the known structure of this microsatellite locus were identified in the database-reported sequence of this BAC clone (accession No. AL591746). The presence of this microsatellite sequence was not detected in an overlapping BAC clone, RP23-305P10.

EUI and HIV-1-Infected Individuals.

42 heterosexual couples discordant for HIV-1 serostatus were enrolled in the present study. In 32 couples the female partner was HIV-1-infected, whereas the male partner was HIV-seronegative despite a prolonged history of penetrative sexual intercourse without condom. In the remaining 10 couples the male partner was HIV-1-infected, whereas the female partner was HIV-seronegative. Inclusion criteria for the EUI group were a history of multiple unprotected sexual episodes for at least 4 years with at least an episode of at-risk intercourse within the 4 months prior to the study period. The couples reported an average of 8 unprotected sexual episodes/year (range 5 to >40) in the 4 years. Forty-nine additional age-and sex-matched HIV-1-infected individuals were enrolled by the Infectious Diseases Unit of the Ospedale Santa Maria Annnunziata, Florence, Italy. Finally, 47 uninfected age-and sex matched healthy healthy control individuals were enrolled as volunteers from the Luigi Sacco Hospital, in Milano, and the Santa Maria Annunziata Hospital in Florence. The Research Ethics Committees of the Luigi Sacco Hospital, Milano, and of the Santa Maria Annunziata Hospital, Florence, have approved this protocol, and genotyping analyses of the enrolees were approved by the Ethics Committee of Kinki University School of Medicine. Written informed consent was obtained from all enrolees before the enrollment, and samples were anonymized and analyzed in a blinded fashion.

Phenotype Definition.

Plasma HIV-1 load was quantified by using the AMPLICOR HIV Monitor test (Roche Diagnostic Systems, Nutley, N.J.) as described previously [21, 23], and was undetectable in all the EUIs and healthy controls. To exclude the possibility of a mucosally-confined presence of HIV-1 in the EUIs, possible presence of HIV-1 cDNA was analyzed in seminal or vaginal fluid by a reverse transcription and PCR method as described [21, 23]. HIV-1 cDNA was not detected in the EUIs. Titration of HIV-1-specific antibodies in serum and urethral or vaginal swabs was performed by an enzyme-linked immunoassay using the HIV EIA test (Calypte Biomedical Corp., Berkeley, Calif.) as described [21-26]. The EUIs possessed no detectable HIV-reactive IgG in their serum, while all the HIV-infected individuals were positive for serum IgG reactive to HIV-1. For the detection and enumeration of HIV-1-ractive memory T-cells in the peripheral blood, an enzyme-linked immunospot (ELISPOT) assay was performed as described previously [23]. In brief, PBMCs were stimulated with a mixture of 5 synthetic peptides representing the immunodominant and promiscuous epitopes identified in the HIV-1 envelope glycoprotein, gp160, and cultured in anti-interferon (IFN)-γ antibody-coated 96-well plates. Spots of secreted IFN-γ were visualized and counted by using a biotin-conjugated anti-IFN-γ antibody (Mabtech, Nacka, Sweden), streptavidin-conjugated alkaline phosphatase (Mabtech), and a phosphatase substrate kit (Bio-Rad Laboratories, Hercules, Calif.).

Analyses of Human SSLP Markers.

500 ng of genomic DNA extracted from PBMCs of each examined individual was used as the template for 40 cycles of PCR amplification using the flanking primer sets synthesized based on the sequence data that are described in the Ensembl Genome Browser). Each left primer was labelled with a florescent dye for detection in fragment analyses with an ABI 3100 DNA sequencer (Applied Biosystems, Foster City, Calif.). PCR amplification was performed with recombinant Taq polymerase (Invitrogen Life Technologies) in the following conditions: initial denaturation at 94° C. for 2 min., each amplification cycle comprising 94° C. for 30 sec., 55° C. for 30 sec., and 74° C. for 90 sec., with final elongation at 74° C. for 10 min. For genotyping each PCR amplified fragment (50-100 fmol) was applied to the above DNA sequencer along with appropriate size markers. Peak identification and size measurements were done with GeneScan software (Applied Biosystems). For determination of absolute fragment sizes, PCR products obtained from at least two homozygous individuals for each examined locus were cloned into pCR2.1-TOPO vector (Invitrogen Life Technologies), and sequenced by using the M13 forward primer. Sequencing was repeated until at least 6 identical clones were observed for each allele.

Statistical Analyses.

Standard statistics used for comparisons of allele frequency distributions and results of immunological assays are specified in the corresponding parts of the text and table notes. To examine possible presence of a dominant allele having different frequencies between the three phenotypic groups, mathematical analyses were performed as follows. Define xᵢⱼ as the number of individuals having the genotype i/j (i ≦j) for the EUI group, where n=Σ_{i≦j} xᵢⱼ is the total number of individuals that belong to this group. Assume that x=(xᵢⱼ)_{i≦j} has a multinomial distribution with the parameter a=(aᵢⱼ)_{i≦j}, where Σ_{i≦j} aᵢⱼ=1. For convenience, let aⱼᵢ=aᵢⱼ. Similarly, we define the notations y, b and z, c for the HIV-1-infected and healthy control groups, respectively. The frequency of the individuals having the allele i for the EUI group is expressed as aᵢ=Σₖ aᵢₖ. Similarly, define bᵢ and cᵢ. A hypothesis where the frequencies of the individuals having the allele i for the EUI and HIV groups is the same is expressed as Hᵢ: aᵢ=bᵢ. Similarly also consider the hypothesis aᵢ=cᵢ to compare the EUI with healthy control groups.

In the present study, we tested multiple hypotheses Hᵢ's and obtained the corresponding statistical P values. It should be noted that even if the judgement for each Hᵢ is done at a preset significance level, the whole judgement may actually be done at a larger significance level, because error rates are accumulated after more than one testing. False rejection of a null hypothesis as above can be overcome by using a closed testing procedure as follows [77]. Let H be the closed set consisting of all the intersections of the hypotheses Hᵢ's. Assume that we can make the reject region with common significant level a for any hypothesis H ∈ H. The closed testing procedure says that we can reject H ∈ H only after we reject all the hypotheses including H, using the corresponding reject region. Thus, the remaining problem is to set the rejection regions.

Let tᵢ be the standardized test statistic for the hypothesis Hᵢ. The corresponding reject region becomes Wᵢ={|tᵢ|>eᵢ}. Consider a common hypothesis H. For example, let H be the intersection of H₁. . . , H_{I}. The corresponding reject region can be defined by W={max_{i=1, . . . ,I} |tᵢ|>e}. We used the following variance stabilizing type as the standardized test statistic:
tᵢ=(sin⁻¹\sqrt{xᵢ/nₓ−}sin⁻¹{square root over (yᵢ/n_{y}))}/\sqrt{¼nₓ+¼n_{y}}
where nₓ=Σ_{i≦j} xᵢⱼ and n_{y}=Σ_{i≦j} yᵢⱼ. As an advantage over the commonly used likelihood ratio and Pearson's X² tests, the above type enables us to interpret that the smaller a P value is the stronger the rejection of the corresponding null hypothesis, because the variances of the arcsine are constant independently of the samples. The above procedure also has another merit: If the maximal intersection hypothesis H ∈H is rejected, the individual hypothesis corresponding to the minimum P value can automatically be rejected in view of the closed testing procedure. In addition, if the hypothesis corresponding to the minimum P value alone is rejected among the individual hypotheses, it is the only rejected hypothesis in view of the closed testing procedure. If the number of alleles is l, there are l hypotheses, Hᵢ's. Consider that corresponding P values for Hi's are p₁< . . . <pₗ for simplicity. The maximal intersection hypothesis is the intersection of Hi's. If this hypothesis is rejected, in other words, if the corresponding P value is less than the common significance level α, the hypothesis H₁ corresponding to the minimum P value can be rejected in view of the closed testing procedure. In addition, if p₁<α<P₂< . . . , the rejected hypothesis is H₁ alone.

The joint distribution of tᵢ's can usually be approximated by the multivariate normal distribution under the null hypothesis, and therefore the corresponding approximated P values can easily be calculated for the individual hypotheses. The approximated P values for a common hypothesis can be calculated by using the central limit theorem and the parametric bootstrap [78]. Note that this procedure is more powerful than simple Bonferroni correction. To avoid unnecessary disturbances due to the presence of alleles having a small frequency, we tested only the hypotheses having the estimated frequency larger than 0.1 when considering a common hypothesis, because alleles with a frequency smaller than 0.1 cannot explain the phenotype of the whole group. Calculations were performed by drawing 100,000 random samples from the approximated multivariate In the present study we have demonstrated that the presence or absence of detectable titres of virus-neutralizing antibodies in FV-infected (B10.A×A)×A backcross mice at 15 days after infection is tightly associated with their genotypes at the chromosome 15 loci. The linkage mapping data indicated that a single gene controlling the production of virus-neutralizing antibodies located near the D15Mit71 locus, co-localizing with the previously mapped Rfv-3 locus [45, 46]. Since the Rfv-3-associated phenotypes were defined by clearance of viraemia by 35-40 days after FV infection [4446], and neutralizing antibodies were detectable at 15 days after infection in mice possessing the B10-derived dominant allele on chromosome 15 (FIG. 4), it is highly conceivable that early production of virus-neutralizing antibodies is associated with early clearance of viraemia. As can be seen in FIG. 1, a, changes in the average titre (n=11-16) of virus-neutralizing antibodies in (B10.A×A)F₁ (●) and A (∘) mice at different time points after FV infection. S.E.M. are shown with the bars. The dashed line indicates the limit of detection. b, titres of virus-neutralizing antibodies in each individual mouse tested at PID 15. Genotypes at the D15Mit17 locus are either homozygous for the A-derived allele (∘) or heterozygous for the B10.A-derived and A-derived alleles (●).

It is most intriguing that genotypes at microsatellite loci located within the segment of human chromosome 22 that is syntenic to mouse chromosome 15 were associated with the presence of mucosal anti-HIV IgA in HIV-1-uninfected individuals. The highest correlation was observed at the D22S423 locus where the frequency of individuals possessing the allele 221 was significantly higher in the EUI group than in the HIV-1-infected one even after corrections for multiple comparisons were made. This marker locus is located in the middle of the chromosomal segment corresponding to the region of mouse chromosome 15 that harbours the gene locus controlling the production of virus-neutralizing antibodies (FIG. 4). In FIG. 4, physical mapping and synteny data are based on those compiled in the Ensembl Genome Browser. Centromeres (∘) are placed on the left. The location of the mouse locus controlling the production of FV-neutralizing antibodies was determined by linkage analyses using 143 (B10.A×A)×A backcross mice, and was further narrowed to the shown area by correlating the SSLP genotypes and antibody titres in the 8 critical animals in which a chromosomal recombination was identified between the. D15Mit105 and D15Mit107 loci. Human SSLP loci at which genotypes were compared between the EUI and HIV-infected groups are also shown. It may also be worth noting that the alleles 156 and 158 at the D22S277 locus that are rare (5.6 and 9.3% per chromosome, respectively) among the database-reported Caucasian CEPH population [59, 60] were more frequently observed in the EUI group (9.5 and 17.9%, respectively). The frequencies of these alleles observed in the HIV-1-infected and healthy control groups of individuals were comparable to those reported fro the CEPH population, and there was significant difference in the frequency of the allele 156 (P=0.035 by two-tailed Fisher's exact test) and that of the allele 158 (P=0.0061 by the same test) when the HIV-1-infected and healthy control groups were combined and compared with the EUI group. Since the rates of microsatellite mutation are much higher than those of point mutation at coding genes [61], and the most common step-wise mutation is biased toward the reduction of repeat numbers for microsatellites of >20 repeats [62], it is justifiable to hypothesize that the alleles 156 and 158 at the D22S277 locus (25 and 26 dinucleotide repeats, respectively) are both linked to a same putative allele that is associated with the enhanced immune responses to HIV-1 in the uninfected individuals. In this regard, the variance stabilizing analyses performed by assuming that the alleles 156 and 158 are both linked to a single dominant genetic factor resulted in the demonstration of significant differences between the EUI and HIV-1-infected, and the EUI and healthy control groups at P=0.0066 and 0.0079, respectively, and these two individual null hypotheses were also rejected (significant difference validated) after the correction for multiple comparisons was made with the closed testing procedure at P=0.0378 and 0.0448, respectively. Further, when the same comparison was made between a combined group of the HIV-1-infected and healthy control individuals and the EUI group, frequency of individuals possessing either the allele 156 or 158 was significantly higher among the EUIs (P=0.0019), and this was strongly significant even after the correction for multiple comparisons was made (P=0.0121). Thus, genotypes at multiple loci within the segment of human chromosome 22 that is syntenic to mouse chromosome 15 are significantly associated with the presence of strong mucosal and T-cell immune responses against HIV-1 in HIV-uninfected Italians.

Production of virus-neutralizing antibodies in FV-infected mice is dependent on CD4⁺ T helper cell functions [63], and the T-cell recognition of the viral envelope epitope(s) strongly influences the kinetics of the class switching of virus-neutralizing antibodies [64,65]. Likewise, HIV-1-exposed and uninfected individuals enrolled into the present study possessed significantly higher amounts of mucosal anti-HIV-1 IgA and larger numbers of HIV-1 envelope-reactive T cells in the peripheral blood in comparison with the HIV-infected individuals (Table 2).

TABLE 2
HIV-1-related phenotypes of the three groups genetically analyzed in
the present study.

| | | | | | HIV-1 |
|---|---|---|---|---|---|
| | | | Urethral/ | | envelope- |
| | | | vaginal anti- | Serum anti- | reactive |
| | | Plasma HIV | HIV-1 IgA | HIV-1 IgG | IFN-γ |
| | | load | (Optical | ((Optical | ELISPOT |
| Group | Age | (copies/ml) | Density) | Density) | (/10⁶ cells) |
| HIV-1- | 40.1 ± 1.4 | Not | 0.556 ± 0.047^{b} | 0.004 ± 0.0006 | 131.2 ± 11.3^{c} |
| exposed and | | detectable | | | |
| uninfected | | (all)^{a} | | | |
| HIV-1- | 40.8 ± 1.9 | 5.0 ± 3.4 × 10⁵ | 0.360 ± 0.039 | 0.793 ± 0.069 | 63.4 ± 9.2 |
| infected | | | | | |
| Healthy | 37.8 ± 3.6 | Not | 0.002 ± 0.0006 | 0.002 ± 0.0001 | <5 |
| control | | detectable | | | |
| | | (all)^{a} | | | |

Numbers are mean ± S.E.M.
^{a}All enrolees were tested for the presence of HIV genome by measuring plasma HIV RNA and by detecting HIV cDNA from total RNA of PBMCs. In the case of the exposed and uninfected individuals, possible presence of HIV cDNA was also tested by PCR of mucosal biopsies. All the individuals in the exposed and uninfected and healthy control groups were negative for all these tests.
^{b}Significantly higher than the average for the HIV-1-infected individuals at P = 0.0022 by Welch's t test.
^{c}Significantly higher than the average for the HIV-1-infected individuals at P = 0.015 by Welch's t test.

Thus, all these data are consistent with the hypothesis that a dominant responder allele of a gene, possibly homologous to the mouse allele that confers the ability to produce retrovirus-neutralizing antibodies at an early stage of FV infection, is present in chromosome 22, and located near the, 22q13.1 segment. None of the previously reported human genes that affect the resistance against HIV-infection and/or the progression to acquired immunodeficiency syndrome are located in this area of the human chromosome, as CCR5 and CCR2 being located at 3p21, SDF1 at 10q11.1, HLA at 6p21.3, KIRs at 19q13.4, and IL10 at 1q31-32 (ref. 3, 38-47). In addition, the CCR5 Δ32 mutation, which results in the lack of cell-surface expression of the HIV co-receptor in homozygotes [20, 66-69], was not found in the enrolees of the present study (data not shown) whereas 3/42 EUIs showed the presence of a heterologous CCR5-Δ32 deletion. This mutation is known to be rare among the HIV-1-exposed and uninfected individuals in Italy and Thailand [21, 26, 74]. Altogether, the present inventor's results indicate the presence of a novel genetic factor which may confer immune resistance against HIV-1 infection.

Linkage mapping of a mouse locus controlling neutralizing antibodies. To exclude the effect of host genes influencing the entry and replication of FV and of those affecting host T-cell responses to the viral antigens, genetic analyses were performed by using crosses of B10.A and A strains of mice which share FV-susceptible Fv-1^{b/b}, Fv-2^{s} (either Fv-2^{r/s} or Fv-2^{s/s}) and H2^{a/a} genotypes. When (B10.A×A)F₁ and A mice were compared for their production of virus-neutralizing antibodies at different time points after FV infection, none of these mice possessed a detectable level of neutralizing antibodies at 10 days after infection. Neutralizing antibodies were kept undetectable at post-infection days (PID) 15 and 20 in parental A mice. In contrast, all the individuals of infected (B10.A×A)F₁ mice possessed a significant neutralizing titre at PID 15, and the titres significantly increased in comparison with those at PID 15 when tested at PID 20 (FIG. 1). Therefore, possible segregation of neutralizing antibody titres in (B10.A×A)×A backcross mice was tested by bleeding them at PIDs 15, 17 and 21. Virus-neutralizing antibodies were not detectable in 63 (44%) of the 143 (B10.A×A)×A backcross mice at PID 15 (FIG. 1), suggesting that a single locus is involved in the production or lack of production of neutralizing antibodies. For linkage analyses, genotyping was concentrated on chromosome 15 because the Rfv-3 locus that influences the persistence of viraemia has been mapped in this chromosome, and initial analyses performed by using 43 separate backcross individuals showed significant correlation between virus-neutralizing titres at PID 17 and genotypes at four loci in the chromosome 15, D15Mit22, D15Mit28, D15Mit42, and D15Mit161 (data not shown). The results of linkage analyses performed by using 143 backcross mice indicated a strong correlation between genotypes at marker loci in chromosome 15 and titres of virus-neutralizing antibodies at PID 15, with the strongest correlation (X²=74.0, P=1.17×10⁻⁷) observed at the D15Mit17 locus (Table 3).

TABLE 3
Mapping of a putative locus controlling the production
of FV-neutralizing antibodies at PID 15.

| Genetic locus | Lod score | X² value |
|---|---|---|
| D15Mit22 | 4.37 | 22.3 |
| D15Mit28 | 13.60 | 62.8 |
| D15Mit71 | 16.38 | 74.0 |
| D15Mit171 | 11.15 | 50.7 |
| D15Mit42 | 7.09 | 31.7 |
| D1Mit48 | 1.79 | 7.7 |
| D2Mit184 | Not linked | 1.4 |
| D14Mit115 | Not linked | 2.7 |

Linkage mapping with MAPMAKER/EXP located a locus determining the presence or absence of virus-neutralizing antibodies at PID 15 between the D15Mit17 and D15Mit171 loci, which is consistent with the previous mapping of the Rfv-3 locus associated with early clearance of viraemia. Further mapping of the locus influencing the production of FV-neutralizing antibodies was performed by genotyping the backcross animals that possessed a critical recombination between the D15Mit28 and D15Mit171 loci. For this purpose approximately 12 Mbp region of chromosome 15 surrounding the D15Mit71 locus was covered with 18 polymorphic microsatellite markers, and their genotypes in each individual backcross mouse were determined. As a result, 8 backcross mice that possessed reciprocal recombination within this region were identified (FIG. 4). Note that, although D15Mit1 is currently not included in the physical map of the mouse genome archived in the Ensembl Genome Browser (hftp://www.ensembl.org/), we identified the flanking primer and repeat sequences of this microsatellite marker within a clone of bacterial artificial chromosome, RP23-290M7, harbouring the segment of mouse chromosome 15 (base numbers 4791548097). Thus, the physical map of the D15Mit1 locus is included in FIG. 4. Since significant correlation (P=0.029 by two-tailed Fisher's exact test) between genotypes at the D15Mit71, D15Mit2, D15Mit214, D15Mit69, and D15Mit70 loci with the production of virus-neutralizing antibodies at PID 15 was observed in these reciprocally recombinant animals, it is conceivable that the locus controlling the production of FV-neutralizing antibodies is located within the region telomeric to the D15Mit1 and centromeric to D15Mit118 loci at the widest.

Genetic Analyses of HIV-1-Exposed and Uninfected Italians.

The present inventors next explored a possibility that a putative ortholog of the above mouse locus might influence antibody production in human retrovirus infections. Because of the rout of transmission of HIV-1 and resultant rarity of multicase families, standard linkage analyses by comparing affected and unaffected siblings are impossible. Therefore, the present inventors performed a simple association study by comparing genotypes between the exposed and uninfected and HIV-1-infected groups of individuals, hypothesizing that presumably protective anti-HIV-1 immune responses are associated with the presence of a dominant genetic factor in the uninfected individuals, and this factor is lacking in the infected individuals. In addition, the present inventors also hypothesized that the above putative genetic factor might be an ortholog of the mouse locus that confers the ability to produce virus-neutralizing antibodies in the early stage of FV infection. Therefore, the present inventors concentrated on polymorphic genetic markers in the segment of human chromosome 22 that is syntenic to mouse chromosome 15 (FIG. 4). 42 uninfected partners of HIV-1-infected individuals who possessed mucosal anti-HIV-1 IgA and exhibited HIV-1 peptide-specific T-cell cytokine production despite undetectable blood and cellular HIV genome, 49 HIV-1-infected individuals including the infected partners of the above EUIs, and 47 uninfected healthy controls were enrolled from Milan and Florence areas in Italy with written informed consent, and were genotyped at the loci shown in FIG. 4. Virological and immunological parameters of the three phenotypic groups are summarized in Table 2. Possible genetic differences between the three groups were compared by two methods. First, distributions of allele frequencies at an examined locus were compared between each pair of the three phenotypic groups using Pearson's X² analyses for contingency tables of 2×(number of alleles). For ethnological interests, allele frequency distributions of the enrolees as a combined group were also compared with those of the CEPH family Caucasians reported in The Genome Database (GDB ver. 6.4). Second, frequencies of individuals possessing an allele in question at a given locus were mathematically compared between the three phenotypic groups using a variance stabilizing statistic as described in the Methods section. Thus, distributions of allele frequencies at the examined loci between the enrolled Italians and the CEPH family Caucasian population were not different except at the D22S423 and D22S1166 loci (P=0.0061 and 0.0087, respectively). When allele frequencies were compared between the three phenotypic groups, their distribution at the D22S277 locus differed between the EUI and healthy control groups at P=0.039. No significant difference was observed at the other loci.

When frequencies of individuals possessing a particular allele at a given locus were compared between the three phenotypic groups by adopting a dominant model, objective mathematical analyses revealed multiple loci with significant differences (Table 4).

TABLE 4
Chromosome 22q association analyses

| | | | Frequency | | P value | |
|---|---|---|---|---|---|---|
| Cytogenetic | | Allele | in the EUI | Compared | Individual | Common |
| location | Locus | size (bp) | group^{a} | to^{b} | hypothesis | hypothesis^{c} |
| 22q11.21 | D22S264 | 188 | 0.405 | HIV | 0.0142 | ns |
| | | 198 | 0.238 | HC | 0.0128 | ns |
| 22q12.2 | D22S277 | 158 | 0.310 | HIV | 0.0152 | ns |
| | | 162 | 0.262 | HC | 0.0371 | ns |
| 22q13.1 | D22S272 | 134 | 0.667 | HC | 0.0243 | 0.0466 |
| 22q13.1 | D22S423 | 221 | 0.333 | HIV | 0.0087 | 0.0317 |
| | D22S1166 | 134 | 0.571 | HC | 0.0266 | ns |
| 22q13.2 | D22S418 | 145 | 0.605 | HC | 0.0475 | ns |
| 22q13.32 | D22S1169 | 126 | 0.643 | HC | 0.0313 | ns |

^{a}Frequencies of individuals possessing the indicated allele on at least one choromosome.
^{b}HIV, HIV-1-infected individuals; HC, healthy control.
^{c}ns, not significant at the P < 0.05 level.

These individual differences were further examined for possible false rejection of a single null (equal frequency) hypothesis due to multiple comparisons by using the closed testing procedure. As a result, frequencies of individuals possessing the allele 134 at the D22S272 locus were significantly different between the EUI and healthy control groups, and those of individuals possessing the allele 221 at the D22S423 locus were also different significantly between the EUI and HIV-infected individuals.

The present inventors have found that the alleles D22S929, D22S272, D22S284 and D2281166 are more frequent in uninfected HIV-exposed patients as shown by [symbol] , [symbol] , and [symbol] in the following table whereas the alleles D22S299 are found less frequently as shown by [symbol] in the following table.

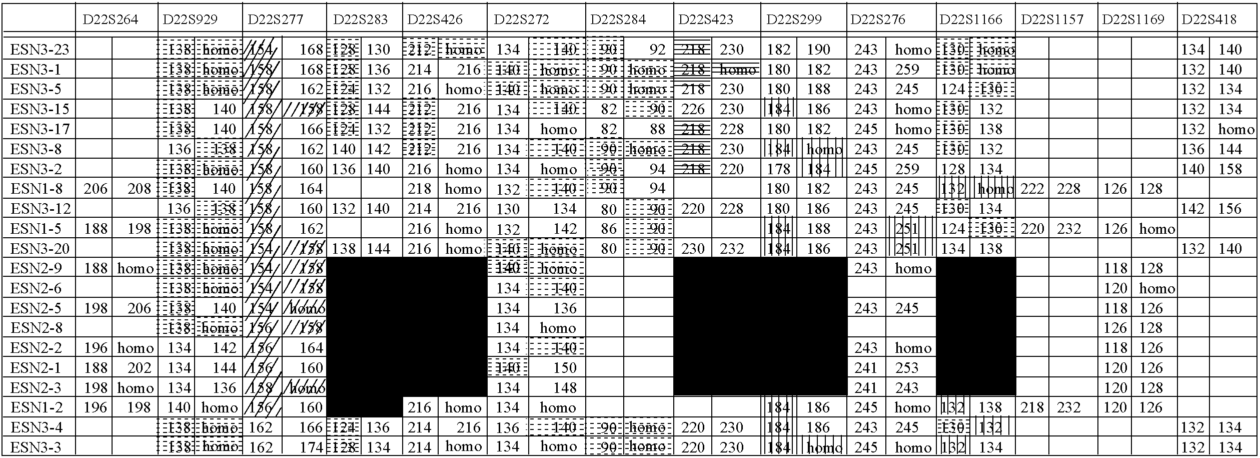

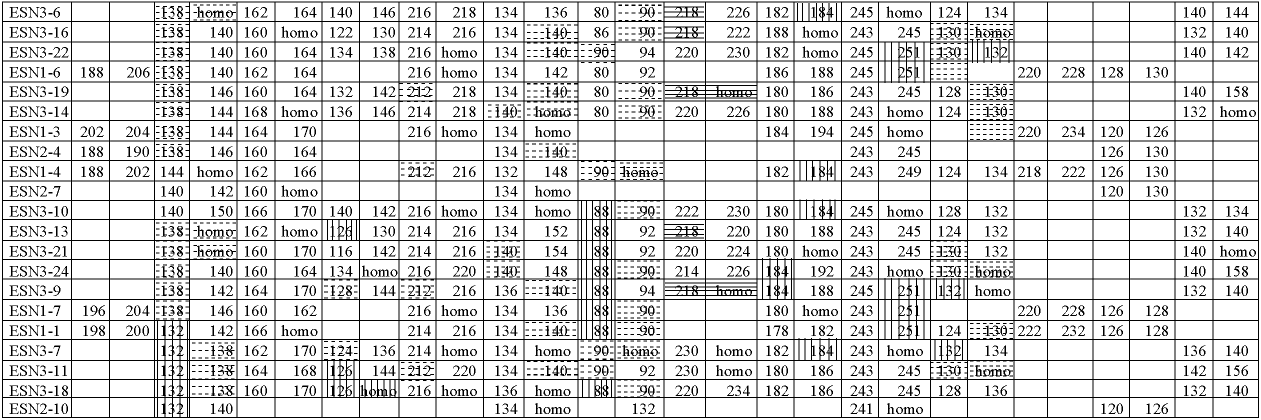

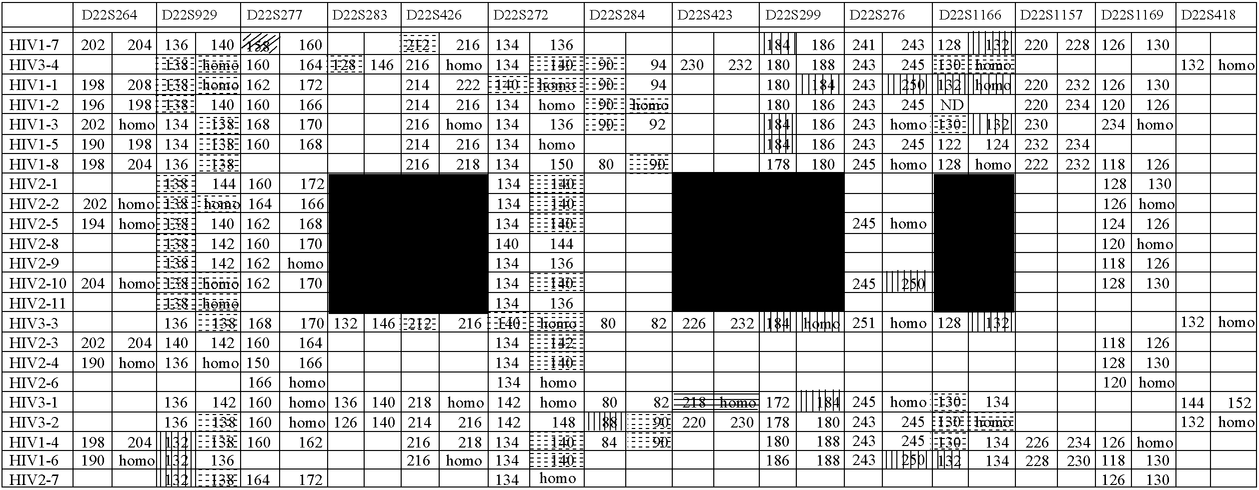

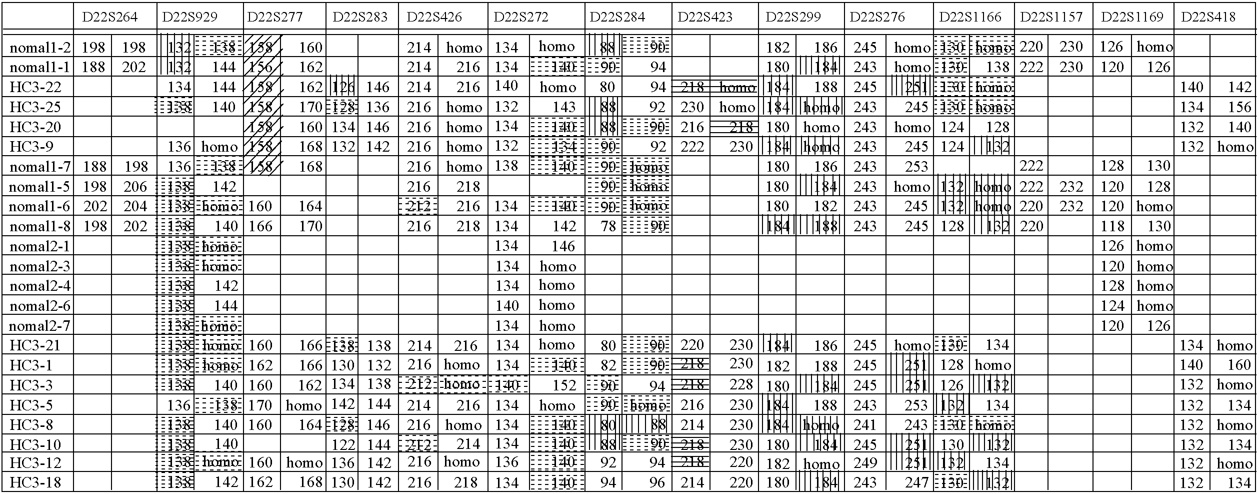

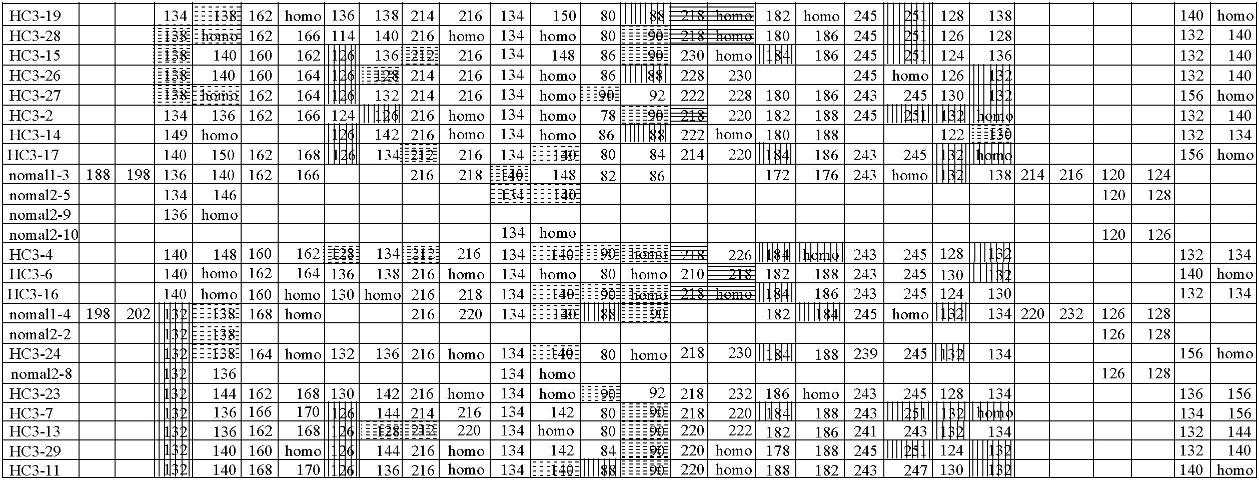

### REFERENCES

1. O'Brien, S. J., Nelson, G. W., Winkler, C. A. & Smith, M. W. Polygenic and multifactorial disease gene association in man: Lessons from AIDS. Annu. Rev. Genet. 34, 563591 (2000).

2. Dean, M. et al. Genetic restriction of HIV-1 infection and progression to AIDS by a deletion allele of the CCR5 structural gene. Science 273, 1856-1862 (1996).

3. Liu, R. et al. Homozygous defect in HIV-1 coreceptor accounts for resistance of some multiply-exposed individuals to HIV-1 infection. Cell 86, 367-377 (1996).

4. Samson, M. et al. Resistance to HIV-1 infection in Caucasian individuals bearing mutant alleles of the CCR-5 chemokine receptor gene. Nature 382, 722-725 (1996).

5. Martin, M. P. et al. Genetic acceleration of AIDS progression by a promoter variant of CCR5. Science 282, 1907-1911 (1998).

6. Smith, M. W. et al. Contrasting genetic influence of CCR2 and CCR5 receptor gene variants on HIV-1 infection and disease progression. Science 277, 959-965 (1997).

7. Winkler, C. et al. Genetic restriction of AIDS pathogenesis by an SDF-1 chemokine gene variant. Science 279, 389-393 (1998).

8. Carrington, M. et al. HLA and HIV: Heterozygote advantage and B*35-Cw*04 disadvantage. Science 283, 1748-1752 (1999).

9. Shin, H. D. et al. Genetic restriction of HIV-1 infection and AIDS progression by promoter alleles of interleukin 10. Proc. Natl. Acad. Sci. USA. 97, 14467-14472 (2000).

10. Beyrer, C. et al. Epidemiologic and biologic characterization of a cohort of human immunodeficiency virus type 1 highly exposed, persistently seronegative female sex workers in northern Thailand. J. Infect. Dis. 79, 59-68 (1999).

11. Martin, M. P. et al. Epistatic interaction between KIR3DS1 and HLA-B delays the progression to AIDS. Nature Genet 31, 429-434 (2002).

12. Rowland-Jones, S. L. & McMichael, A. Immune responses in HIV-exposed seronegatives: Have they repelled the virus? Curr. Opin. Immunol. 7, 448-455 (1995).

13. Shearer, G. M. & Clerici, M. Protective immunity against HIV infection: Has nature done the experiment for us? Immunol. Today 17, 21-24 (1996).

14. Mazzoli, S. et al. HIV-specific mucosal and cellular immunity in HIV-seronegative partners of HIV-seropositive individuals. Nature Med. 3, 1250-1257, 1997.

15.Kaul, R. et al. HIV-1 specific mucosal IgA in a cohort of HIV-1-resistent Kenyan sex workers. AIDS 13, 23-29 (1999).

16. Biasin, M. et al. Mucosal and systemic immune activation is present in human immunodeficiency virus exposed seronegative women. J. Infect Dis. 182,1365-1374 (2000).

17. Belec, L. et al. Cervicovaginal secretory antibodies to HIV type 1 that block viral transcytosis through epithelial barriers in highly exposed HIV-1-seronegative African women. J. Infect. Dis. 184, 1412-1422 (2001).

18. Rowland-Jones, S. L. & McMichael, A. Immune responses in HIV-exposed seronegatives: Have they repelled the virus? Curr. Opin. Immunol. 7, 448-455 (1995).

19. Shearer, G. M. & Clerici, M. Protective immunity against HIV infection: Has nature done the experiment for us? Immunol. Today 17, 21-24 (1996).

20. O'Brien, S. J., Nelson, G. W., Winkler, C. A. & Smith, M. W. Polygenic and multifactorial disease gene association in man: Lessons from AIDS. Annu. Rev. Genet. 34, 563-591 (2000).

21. Mazzoli, S. et al. HIV-specific mucosal and cellular immunity in HIV-seronegative partners of HIV-seropositive individuals. Nature Med. 3, 1250-1257 (1997).

22. Kaul, R. et al. HIV-1 specific mucosal IgA in a cohort of HIV-1-resistant Kenyan sex workers. AIDS 13, 23-29 (1999).

23. Biasin, M. et al. Mucosal and systemic immune activation is present in human immunodeficiency virus-exposed seronegative women. J. Infect. Dis. 182, 1365-1374 (2000).

24. Mazzoli, S. et al. Human immunodeficiency virus (HIV)-specific IgA and HIV neutralizing activity in the serum of exposed seronegative partners of HIV-seropositive persons. J. Infec. Dis. 180, 871-875 (1999).

25. Belec, L. et al. Cervicovaginal secretory antibodies to HIV type 1 that block viral transcytosis through epithelial barriers in highly exposed HIV-1-seronegative African women. J. Infect. Dis. 184, 1412-1422 (2100).

26. Locaputo, S. et al. Mucosal and systemic HIV-specific immunity in HIV-exposed but uninfected heterosexual males. AIDS 17, 531-538 (2002).

27. Letvin, N. et al. Potent, protective anti-HIV immune responses generated by bimodal HIV envelope DNA plus protein vaccination. Proc. Natl. Acad. Sci. USA 94, 9378-9383 (1997).

28. Mascola, J. R. et al. Protection of macaques against pathogenic simian/human immunodeficiency virus 89.6PD by passive transfer of neutralizing antibodies. J. Virol. 73, 4009-4018 (1998).

29. Mascola, J. R . et al. Protection of macaques against vaginal transmission of pathogenic HIV-1/SIV chimeric virus by passive infusion of neutralizing antibodies. Nat Med. 6, 207-210 (2000).

30. Earl, P. L. et al. Immunogenicity and protective efficacy of oligomeric human immunodeficiency virus type 1 gp140. J. Virol. 75, 645-653 (2001).

31. Teich, N., Wyke, J., Mak, T., Bernstein, A. & Hardy, W. Pathogenesis of retrocirus-induced disease. RNA Tumor Viruses, 2^{nd} ed. Cold Spring Harbor Laboratory, New York (19872), pp785-998.

32. Kabat, D. Molecular biology of Friend viral erythroleukemia. Curr. Top. Microbiol. Immunol. 148,142 (1989).

33. Chesebro, B., Miyazawa, M. & Britt, W. J. Host genetic control of spontaneous and induced immunity to Friend murine retrovirus infection. Annu. Rev. Immunol. 8, 477-499 (1990).

34. Hoatlin, M. E. & Kabat, D. Host-range control of a retroviral disease: Friend erythroleukemia. Trends Microbiol. 3, 51-57 (1995).

35. Best, S., Le Tissier, P. Towers, G. & Stoye, J. P. Positional cloning of the mouse retrovirus restriction gene Fv1. Nature 382, 826-829 (1996).

36. Persons, D. A. et al. Fv2 encodes a truncated form of the Stk receptor tyrosine kinase. Nat. Genet. 23, 159-165 (1999).

37. Ikeda, H., Laigret, F., Martin, M. A. & Repaske, R. Characterization of a molecularly cloned retroviral sequence associated with Fv-4 resistance. J. Virol. 55, 768-777 (1985).

38. Ikeda, H. & Sugimura, H. Fv-4 resistance gene: a truncated endogenous murine leukaemia virus with ecotropic interference properties. J. Virol. 63, 5405-5412 (1989).

39. Miyazawa, M., Nishio, J. & Chesebro, B. Genetic control of T cell responsiveness to the Friend murine leukaemia virus envelope antigen. Identification of the class II loci of H-2 as immune response genes. J. Exp. Med. 168, 1587-1605 (1988).

40. Iwashiro, M. et al. Multiplicity of virus-encoded helper T-cell epitopes expressed on FBL-3 tumor cells. J. Virol 67, 4533-4542 (1993).

41. Peterson, K. E., Iwashiro, M., Hasenkrug, K. J. & Chesebro, B. Major histocompatibility complex class I gene controls the generation of gamma interferon-producing CD4⁺ and CD8⁺ T cells important for recovery from Friend retrovirus-induced leukaemia. J. Virol. 74, 5363-5367 (2000).

42. Miyazawa, M., Nishio, J., Wehrly, K., David, C. S. & Chesebro, B. Spontaneous recovery from Friend retrovirus-induced leukaemia. Mapping of the Rfv-2 gene in the Q/TL region of mouse MHC. J. Immuno. 148, 1964-1976 (1992).

43. Iwanamil, N., Niwa, A., Yasutomi, Y., Tabata, N. & Miyazawa, M. Role of natural killer cells in resistance against Friend retrovirus-induced leukaemia. J. Virol. 75, 3152-3163 (2001).

44. Chesebro, B. & Wehrly, K. Studies on the role of the host immune response in recovery from Friend virus leukaemia. I. Antiviral and antileukemia cell antibodies. J. Exp. Med. 143, 73-84 (1976).

45. Hasenkrug, K. J. et al. Chromosome mapping of Rfv3, a host resistance gene to Friend murine retrovirus. J. Virol. 69, 2617-2620 (1995).

46. Super, H. J. et al. Fine mapping of the Friend retrovirus resistance gene, Rfv3, on mouse chromosome 15. J. Virol. 73, 7848-7852 (1999).

47. Doig, D. & Chesebro, B. Anti-Friend virus antibody is associated with recovery from viremia and loss of viral leukaemia cell-surface antigens in leukemic mice. Identification of Rfv-3 as a gene locus influencing antibody production. J. Exp. Med. 150, 10-19 (1979).

48. Locaputo, S. et al. Mucosal and systemic HIV-specific immunity in HIV-exposed but uninfected heterosexual males. AIDS, in press (2002).

49. Chesebro, B. & Wehrly, K. Studies on the role of the host immune response in recovery from Friend virus leukaemia. I. Antiviral and antileukemia cell antibodies. J. Exp. Med. 143, 73-84 (1976).

50. Chesebro, B., Miyazawa, M. & Britt, W. J. Host genetic control of spontaneous and induced immunity to Friend murine retrovirus infection. Annu. Rev. Immunol. 8, 477-499 (1990).

51. Hasenkrug, K. J. et al. Chromosome mapping of Rfv3, a host resistance gene to Friend murine retrovirus. J. Virol. 69, 2617-2620 (1995).

52. Super, H. J. et at. Fine mapping of the Friend retrovirus resistance gene, Rfv3, on mouse chromosome 15. J. Virol. 73, 7848-7852 (1999).

53. Miyazawa, M., Nishio, J. & Chesebro, B. Genetic control of T cell responsiveness to the Friend murine leukaemia virus envelope antigen. Identification of the class Il loci of H-2 as immune response genes. J. Exp. Med. 168, 1587-1605 (1988).

54. Miyazawa, M., Nishio, J., Wehrly, K. & Chesebro, B. Influence of MHC genes on spontaneous recovery from Friend retrovirus-induced leukaemia. J. Immunol. 148, 644-646 (1992).

55. Mazzoli, S. et al. Human immunodeficiency virus (HIV)-specific IgA and HIV neutralizing activity in the serum of exposed seronegative partners of HIV seropositive persons. J. Infect Dis. 180, 871-875 (1999).

56. Miyazawa, M., Nishio, J. & Chesebro, B. Protection against Friend retrovirus-induced leukaemia by recombinant vaccinia viruses expressing the gag gene. J. Virol. 66, 4497-4507 (1992).

57. Miyazawa, M. et al. Immunization with a single T helper cell epitope abrogates Friend virus-induced early erythroid proliferation and prevents late leukaemia development. J. Immunol. 155, 748-758 (1995).

58. Weissenbach, J. A second-generation linkage map of the human chromosome. Nature 359, 794-801 (1992).

59. Weissenbach, J. et al. A second-generation linkage map of the human genome. Nature 359, 794-801 (1992).

60. Gyapay, G. et al. The 1993-94 Genethobon human genetic linkage map. Nature Genet 7, 246-339 (1994).

61. Li, Y.-C. et al. Microsatellites: genetic distribution, putative functions and mutational mechanisms: a review. Mol. Ecol. 11, 2453-2465 (2002).

62. Whittaker, J. C. et al. Likelihood-based estimation of microsatellite mutation rates. Genetics 164, 781-787 (2003).

63. Super, H. J., Brooks, D., Hasenkrug, K. & Chesebro, B. Requirement for CD4⁺ T cells in the Friend murine retrovirus neutralizing antibody response: evidence for functional T cells in genetic low-recovery mice. J. Virol. 72, 9400-9403 (1998).

64. Miyazawa, M., Nishio, J., Wehrly, K & Chesebro, B. Influence of MHC genes on spontaneous recovery from Friend retrovirus-induced leukemia. J. Immunol. 148, 644-646 (1992).

65. Miyazawa, M. et al. Immunization with a single T helper cell epitope abrogates Friend virus-induced early erythroid proliferation and prevents late leukemia development. J. Immunol. 155, 748-758 (1995).

66. Dean, M. et al. Genetic restriction of HIV-1 infection and progression to AIDS by a deletion allele of the CCR5 structural gene. Science 273, 1856-1862 (1996).

67. Liu, R. et al. Homozygous defect in HIV-1 coreceptor accounts for resistance of some multiply-exposed individuals to HIV-1 infection. Cell 86, 367-377 (1996).

68. Samson, M. et al. Resistance to HIV-1 infection in Caucasian individuals bearing mutant alleles of the CCR-5 chemokine receptor gene. Nature 382, 722-725 (1996).

69. Martin, M. P. et al. Genetic acceleration of AIDS progression by a promoter variant of CCR5. Science 282, 1907-1911 (1998).

70. Smith, M. W. et at Contrasting genetic influence of CCR2 and CCR5 receptor gene variants on HIV-1 infection and disease progression. Science 277, 959-965 (1997).

71. Winkler, C. et al. Genetic restriction of AIDS pathogenesis by an SDF-1 chemokine gene variant. Science 279, 389-393 (1998).

72. Carrington, M. et al. HLA and HIV: Heterozygote advantage and B*35-Cw*04 disadvantage. Science 283, 1748-1752 (1999).

73. Shin, H. D. et al. Genetic restriction of HIV-1 infection and AIDS progression by promoter alleles of interleukin 10. Proc. Natl. Acad. Sci. USA. 97, 14467-14472 (2000).

74. Beyrer, C. et al. Epidemiologic and biologic characterization of a cohort of human immunodeficiency virus type 1 highly exposed, persistently seronegative female sex workers in northern Thailand. J. Infect Dis. 79, 59-68 (1999).

75. Martin, M. P. et al. Epistatic interaction between KIR3DS1 and HLA-B delays the progression to AIDS. Nature Genet 31, 429-434 (2002).

76. Robertson. M. N., et al. Production of monoclonal antibodies reactive with a denatured form of the Friend murine leukaemia virus gp70 envelope glycoprotein: use in a focal infectivity assay, immunohistochemical studies, electron microscopy and Western blotting. J. Virol. Methods 34, 255-271 (1991).

77. Hsu, J. C. Multiple comparisons: theory and methods. Chapman & Hall/CRC, New York (1996), 277 pp.

78. Hall, P. The bootstrap and edgeworth expansion. Springer, New York (1992), 352 pp.

1. A method of determining a predisposition to infection, the method comprising the steps of obtaining a DNA bearing sample from a subject, and assaying the sample to identify the alleles present at at least one of the microsatellite loci D22S929, D22S277, D22S264, D22S423 & D22S418 or D22S272, wherein the presence of particular alleles at microsatellite loci D22S929, D22S277, D22S264, D22S423 & D22S418 or D22S272 is indicative of a resistance to infection.

2. (canceled)

3. The method according to claim 1, in which the sample is assayed to determine the presence of homologues, splice variants, or derivatives of the microsatellite loci D22S929, D22S277, D22S264, D22S423 & D22S418 or D22S272 or a nucleic acid complementary thereto.

4. The method according to claim 1, in which the infection is viral infection.

5. The method according to claim 4, in which the virus is selected from the group consisting of oncoviruses, retroviruses, lentiviruses, and spumaviruses.

6. The method according to claim 4, in which the virus is an endogenous retrovirus.

7. The method according to claim 3, in which the virus is the HIV virus.

8. The method according to claim 1, in which the sample is obtained non-invasively.

9. The method according to claim 1, in which the sample is blood, urine, semen, mouth swabs, skin cells, nail clippings, hair, high vaginal swabs or a cervical smear.

10. The method according to claim 1, in which the sample is amplified by the use of a nucleic acid amplification technique.

11. The method according to claim 10, in which the nucleic acid amplification technique is PCR or rolling circle replication.

12. The method according to claim 1, in which the sample is assayed for the presence or absence of particular genotypes at the microsatellite locus or loci using DNA fragment length analysis, DNA hybridisation techniques, DNA sequence identification, single strand length polymorphism (SSLP) analysis, or reference strand conformation (RSC) analysis.

13. The method according to claim 12, in which the assay uses single strand length polymorphism (SSLP) analysis and a flanking primer set for PCR amplification of the microsatellite marker is selected from
(SEQ ID No:1)
D22S277 left, TTCTTGTGTGGTAGTCTGGG;
(SEQ ID No:2)
D225277 right, TACCNACTCCCCAAACTATG;
(SEQ ID No:3)
D22S272 left, GAGTTTTGTTTGCCTGGCAC;
(SEQ ID No:4)
D22S272 right, AATGCACGACCCACCTAAAG;
(SEQ ID No:5)
D22S276 left, CATTCTGCCAAGCAATTTAT;
(SEQ ID No:6)
D22S276 right, GCTGCTCTTTAAGTTTCTTGACC;
(SEQ ID No:7)
D22S929 left, GGAGCTGCATGTACTAGCTGG;
(SEQ ID No:8)
D225929 right, GCATTTATGGAGTATCCACAG;
(SEQ ID No:9)
D2251169 left, GCACACACATGCACATAATC;
and
(SEQ ID No:10)
D2251169 right, AACAACTTCCAGCAGACG.
and
complementary nucleic acids or fragments, polymorphisms, splice variants or homologues thereof.

14. A kit for the diagnosis of a predisposition to infection, the kit comprising reagents for determination of genotype at at least one of the microsatellite loci D22S929, D22S277, D22S264, D22S423 & D22S418 or D22S272 or a nucleic acid complementary thereto, fragments, polymorphisms, splice variants or homologues thereof.

15. (canceled)

16. A vector bearing ea chromosomal fragment that comprises at least one of the microsatellite loci D22S929, D22S277, D22S264, D22S423 & D22S418 or D22S272 or a nucleic acid complementary thereto, or fragments, polymorphisms, splice variants or homologues thereof.

17. A method of treating a subject having a predisposition to infection comprising administering to said subject a gene therapy comprising a vector according to claim 16.

18-21. (canceled)

22. A peptide, polypeptide, protein or glycosylation, sulphonation, acetylation, or other post-translational derivative, functional derivative, homologue or fragment thereof comprising an amino acid sequence encoded by a gene located in a chromosomal segment adjacent to microsatellite loci D22S929, D22S277, D22S264, D22S423 & D22S418 or D22S272, complementary nucleic acids or fragments, polymorphisms, splice variants, complementary nucleic acids or homologues of said gene.

23-25. (canceled)

26. A pharmaceutical composition the peptide, polypeptide, protein or glycosylation, sulphonation, acetylation, or other post-translational derivative, functional derivative, homologue or fragment thereof according to claim 22 and a pharmaceutically acceptable carrier.

27. (canceled)

28. (canceled)

29. A contraceptive comprising the peptide polypeptide protein or glycosylation, sulphonation, acetylation, or other post-translational derivative, functional derivative, homologue or fragment thereof according to claim 22.

30. (canceled)

31. A microbicide comprising the peptide, polypeptide, protein or glycosylation, sulphonation, acetylation, or other post-translational derivative, functional derivative, homologue or fragment thereof according to claim 22.

32-34. (canceled)

35. A method for the treatment or prophylaxis of infection comprising administering to a subject in need thereof a therapeutically effective amount of the peptide, polypeptide, protein or glycosylation, sulphonation, acetylation, or other post-translational derivative, functional derivative, homologue or fragment thereof according to claim 22.

36. (canceled)

37. (canceled)

38. A vaccine comprising at least one naked DNA sequence according to any one of SEQ ID NOS:1 to 10.

39. A vaccine comprising a DNA sequences comprising a microsatellite loci D22S929, D22S277, D22S264, D22S423 & D22S418 or D22S272, or fragments, polymorphisms, splice variants, complementary nucleic acids or homologues of said DNA sequence.

40. The vaccine according to claim 39 in which the DNA is naked.

41-43. (canceled)

44. A chip or assay plate comprising DNA encoding the microsatellite loci D22S929, D22S277, D22S264, D22S423 & D22S418 or D22S272, or fragments, polymorphisms, splice variants, complementary nucleic acids or homologues of DNA.

45. A screening assay for identifying a compound that is able to bind to or otherwise recognize DNA encoding the microsatellite loci D22S929, D22S277, D22S264, D22S423 & D22S418 or D22S272, or fragments, polymorphisms, splice variants, complementary nucleic acids or homologues of said DNA, comprising contacting a test compound with the chip or assay plate according to claim 44 and determining whether the compound binds to or otherwise recognizes said DNA.

46. (canceled)

47. A chip or assay plate comprising the peptide, polypeptide, protein or glycosylation, sulphonation, acetylation, or other post-translational derivatives, functional derivatives, homologues or fragments according to claim 22.

48. A screening assay for identifying a compound able to bind to or otherwise recognise, modify or mimic a peptide, polypeptide, protein or glycosylation, sulphonation, acetylation, or other post-translational derivatives, functional derivatives, homologues or fragments of the protein encoded by a gene located in a chromosomal segment adjacent to the microsatellite loci D22S929, D22S277, D22S264, D22S423 & D22S418 or D22S272, complementary nucleic acids or fragments, polymorphisms, splice variants or homologues of said gene, comprising contacting a test compound with the chip or assay plate of claim 47.

49. (canceled)

50. A method for producing an immunoglobulin A which provides resistance to infection or possesses antiviral activity comprising contacting a cell with microsatellite loci D22S929, D22S277, D22S264, D22S423 & D22S418 or D22S272, complementary nucleic acids or fragments, polymorphisms, splice variants or homologues thereof to thereby produce an immunoglobulin A which provides resistance to infection or possesses antiviral activity.

51. The method according to claim 50, further comprising contacting said cell with a peptide, polypeptide, protein or glycosylation, sulphonation, acetylation, or other post-translational derivatives, functional derivatives, homologues or fragments of the protein encoded by the gene located in the chromosomal segment adjacent to the microsatellite loci D22S929, D22S277, D22S264, D22S423 & D22S418 or D22S272, complementary nucleic acids or fragments, polymorphisms, splice variants or homologues of said gene.

52. An Immunoglobulin A providing resistance to infection or possessing antiviral activity, produced according to claim 50.

53. A pharmaceutical composition comprising an immunoglobulin A according to claim 52 and a pharmaceutically acceptable carrier.

54. A method for the treatment of infection comprising administering to a subject in need thereof a therapeutically effective amount of an immunoglobulin A according to claim 52.

55. (canceled)

56. (canceled)

57. The method according to claim 54 produce further comprising producing a mucosal response in said subject undergoing treatment to thereby produce protective immunity in said subject.

58. A mucosal vaccine comprising an immunoglobulin A according to claim 52.

59. A method for producing antigen or pathogen specific immunity in a subject comprising administering to a subject an effective amount of a vaccine according to claim 58.

60. (canceled)

61. A nucleic acid encoding a locus controlling production of neutralising antibodies to HIV.

62. The nucleic acid according to claim 61, in which the nucleic acid is DNA.

63. The nucleic acid according to claim 61, in which the locus is syntenic with mouse D15Mit71.

64. The nucleic acid according to claim 61, in which the locus is selected from human D22S272, D22S423, D22S284, D22S299.

65. The nucleic acid according to claims 61, in which the locus is D22S272 or D22S423.

66. (canceled)

67. A method for the treatment or prophylaxis of HIV infection comprising administering to a subject in need thereof a therapeutically effective amount of a medicament produced from the nucleic acid according to claim 61.

68. A peptide, polypeptide, or protein encoded by a nucleic acid according claim 61.

69. (canceled)

70. A method for the treatment or prophylaxis of HIV infection comprising administering to a subject in need thereof a therapeutically effective amount of a peptide, polypeptide, or protein according to claim 68.
